(19) United States
(12) Patent Application Publication
(10) Pub. No.: US 2026/0158121 A1
(43) Pub. Date: Jun. 11, 2026

(54) TREATMENT OF A HEADACHE DISORDER WITH A BOTULINUM NEUROTOXIN A

(71) Applicant: IPSEN BIOPHARM LIMITED, Wrexham (GB)

(72) Inventors: Laurent Maxime Hubert PONS, Wrexham (GB); Catherine Ruth THOMPSON, Wrexham (GB)

(21) Appl. No.: 19/391,889

(22) Filed: Nov. 17, 2025

Related U.S. Application Data
(63) Continuation of application No. PCT/GB2024/051293, filed on May 17, 2024.

(30) Foreign Application Priority Data
May 18, 2023 (GB) 2307439.6

(51) Int. Cl. A61K 38/48; A61K 9/00; A61P 25/06

(52) U.S. Cl. CPC A61K 38/4893; A61K 9/0019; A61P 25/06; C12Y 304/24069

(57) ABSTRACT

The present invention is directed to the treatment of a headache disorder using botulinum neurotoxin A (BoNT/A).

### CROSS-REFERENCE TO RELATED APPLICATIONS

This application is a Continuation Application of International Application No. PCT/GB2024/051293, filed on May 17, 2024, which claims priority to, and the benefit of Great Britain Application No. 2307439.6, filed on May 18, 2023, the contents of each of which are hereby incorporated by reference in their entireties.

### INCORPORATION BY REFERENCE OF SEQUENCE LISTING

The instant application is being filed with an electronically filed Sequence Listing in XML format. The sequence listing file entitled ST26 sequence listing FINAL-P75069WO.xml, was created on May 15, 2024, and is 10,025 bytes in size. The entire contents of the Sequence Listing are herein incorporated by reference.

### FIELD OF THE INVENTION

The present invention relates to the treatment of a headache disorder.

### BACKGROUND

A headache (medically known as cephalgia) is a condition of mild to severe pain in the head; sometimes neck or upper back pain may also be interpreted as a headache. It may indicate an underlying local or systemic disease or be a disorder in itself. There are multiple different types of headache disorders, including: muscular/myogenic headache, tension headache, vascular headache (e.g. migraine), high blood pressure headache, traction and inflammatory headache (which are usually symptoms of other disorders, ranging from stroke to sinus infection), hormone headache, rebound headache, chronic sinusitis headache, organic headache, and ictal headache (associated with seizure activity).

A key symptom of a headache disorder is typically pain. Pain is an unpleasant sensory and emotional experience associated with, or resembling that associated with, actual or potential tissue damage. Pain is also described as a neurologic condition characterised by pathologic changes in the nervous system or, more precisely, a dysfunction of the endogenous nociceptive system (Raffaeli & Arnaudo (2017), J Pain Res, 10, 2003-2008).

Nociception is the process by which information about actual tissue damage (or the potential for such damage, should the noxious stimulus continue to be applied) is relayed to the brain. The sensory neurons involved in nociception are classified into three main groups: Group A; Group B; and Group C (Yam et al (2018), Int J Mol Sci, 19, 8, 2164).

Group A nerve fibers are classified as myelinated fibers and can be further subdivided into Aα, Aβ, Aγ and Aδ, each with different sets of characteristics. These fibers generally terminate in laminae I, III, IV and V of the dorsal horn of the spinal cord with some lamina II inner projection. Both Type Ia and Ib sensory fibers from muscle spindle endings and Golgi tendons are type Aα. Type Aβ fibers are typically low-threshold, cutaneous, slow or fast adapting mechanoreceptors, and include Type II afferent fibers from the stretch receptor. The Aβ-fibers typically belong to laminae III and IV. Type Aγ fibers may include Type II afferent fibers from the stretch receptors. Type Aδ fibers may include the thermal and mechanical nociceptors that terminate in the Rexed laminae I and V, as well as Type III afferent fibers. Aδ-fibers are also typically the smallest myelinated nerves and may have a relatively fast conduction velocity of ˜30 m/s. The diameter of Aδ-fibers is typically about 2-5 μm and is typically responsive towards short-lasting and pricking pain.

Group B nerve fibers are moderately myelinated usually with conduction velocities of 3-14 m/s. The preganglionic nerve fibers of the autonomous nervous system (ANS) and general visceral afferent fibers belong to this group.

Group C nerve fibers are unmyelinated and are typically less than 2 μm in diameter and have a relatively slow conduction velocity typically of up to approximately 2 m/s. The nerve fibers at the dorsal roots (Type IV afferent fibers) and postganglionic fibers in the ANS may be categorized in this group. All these fibers are mainly nociceptive in function, carrying the sensory information and assembling around 70% of the afferent nociceptive information, which then enters the spinal cord. C-fibers may terminate in laminae I and II in the grey matter of the spinal cord. In terms of nociception, C-fiber nociceptors may be polymodal, as they are activated by thermal, mechanical, and/or chemical stimuli. For example, C-fibers may be activated via poorly localized stimuli. In terms of neurochemistry, C-fibers can be classified as either peptidergic or non-peptidergic, and about 50% of these fibers express neuropeptides including calcitonin gene-related peptide (CGRP), neurokinins and substance P (SP).

There are a variety of neurotransmitters involved in pain, including all the major types of neurotransmitters, such as inflammatory mediators: prostaglandin E2 (PGE2), prostacyclin (PGI2), leukotriene B4 (LTB4), nerve growth factor (NGF), protons, bradykinin (BK), ATP, adenosine, SP, neurokinin A (NKA), neurokinin B (NKB), 5-hydroxytryptamine (5-HT), histamine, glutamate, norepinephrine (NE) and nitric oxide (NO); and non-inflammatory mediators: CGRP, γ-aminobutyric acid (GABA), opioid peptides, glycine and cannabinoids (Yam et al (2018), Int J Mol Sci, 19, 8, 2164).

Of particular therapeutic interest is CGRP, which is widely produced in both the central and peripheral nervous systems; however, it is primarily located in the primary afferent nerves. As a direct derivative of the dorsal root ganglia (DRG), CGRP may be found in the dorsal horn of the spinal cord and associated with the conduction of noxious stimulation. CGRP is related to the excitatory effects of SP, which results in Ca²⁺ release. The receptors of CGRP (calcitonin receptor-like receptor (CALCRL)) are typically located in the nucleus accumbens, indicating that the CNS may control CGRP-mediated pain transmission. CGRP is widely distributed in the peripheral and central nervous system and its receptors are expressed in pain pathways. CGRP-like immunoreactivity (CGRP-LI) is typically found in 40-50% of DRG neurons. Moreover, CGRP is usually co-localized with other neuropeptides, including SP and neurokinins in DRG neurons. Peripheral CGRP-LI fibers may terminate in lamina I, III and V of spinal cord and CGRP-containing DRG neurons innervate joints. Thus, CGRP and its receptors may be widely distributed in peripheral and central pain pathways (Schou et al (2017), The Journal of Headache and Pain, 18, 34, 1-17). In animals, CGRP may be released from peripheral and central nerve endings upon noxious pain and/or mechanical stimulation of the skin. In rats, the major part of circulating CGRP may be released from perivascular nerve terminals. Acute and chronic nociception may lead to altered release of CGRP from sensory nerve endings and central terminals into the dorsal horn of the spinal cord. CGRP is known as one of the most potent vasodilators. Two isoforms have been characterized: α-CGRP and β-CGRP (Russell et al (2014), Physiol Rev, 94, 4, 1099-1142). The isoform α is principally expressed in primary sensory neurons, whereas the isoform β is mainly found in intrinsic enteric neurons. The mature form of this neuropeptide is composed of 37 amino acids, and its expression has been particularly noticed in sensory neurons of the DRG and trigeminal ganglion. The mature form is stored in vesicles localized in the terminal region of central and peripheral nerve endings from where it may be secreted in the dorsal spinal cord or in various peripheral tissues, especially surrounding blood vessels which may modulate vascular tone. In addition, the presence of networks of nociceptors positive to CGRP in rodent and human meningeal vessels has been observed, and about 40-50% of trigeminal ganglion neurons have been found to be positive to CGRP. Moreover, CGRP expression has been observed in areas of the CNS, such as the hypothalamus, thalamus, periaqueductal grey, superior and inferior colliculi, amygdala, trigeminocervical complex, and the cerebellum. These mentioned brain areas may be associated with migraine pathophysiology, considering the capability of CGRP to change synaptic and neuronal activity at the trigeminocervical complex, and transmission of nociceptive signals to the thalamus and cortical areas (Tardiolo et al (2019), Int J Mol Sci, 20(12), 2932).

Conventional treatments for pain include monoclonal antibodies and small-molecule antagonists that target mediators (e.g. pain mediators) once said mediators have already been released by the pre-synaptic neurons. As an alternative approach, certain conventional therapeutics target receptors of the mediators (e.g. pain mediators). These approaches are associated with a number of disadvantages, including: effects on chemical mediators (e.g. CGRP) systemically; nausea; vomiting; dyspepsia; diarrhoea; bradycardia; hypotension; bronchospasm; dyspnoea; fatigue; insomnia; dizziness; dry mouth; flushing; hot or cold sensations; chest pain; constipation; itchiness; drowsiness; ringing in the ears; restlessness; muscle spasms; injection site pain; upper respiratory infection; fatigue; nasopharyngitis; injection site erythema; injection site induration; anxiety; depression; injection site pruritus; influenza; urinary tract infection; somnolence; paraesthesia; increased heart rate; stroke; and/or heart attack (Woo (2020), Nature, 586, S4-S6 and Tardiolo et al (2019), Int J Mol Sci, 20(12), 2932). There is thus a need for improved therapeutics that are associated with fewer side-effects and/or which block mediators (e.g. pain mediators) at the point of release.

Of the different headache disorders, migraine, in particular, is a disabling condition that continues to be an important public health problem. In the United States (US), it accounted for roughly 4 million emergency department (ED) visits in 2016, when headache was the fifth most common reason for an ED visit overall and the third most common reason for ED visits in females aged 15-64. The age-adjusted prevalence of migraine and severe headache in the USA has remained stable over many years. In 2018, the age-adjusted prevalence was 15.9% across all adults. The sex ratio also remains stable, with 21% of women and 10.7% of men affected by migraine.

Migraine is typically diagnosed and classified according to the International Classification of Headache Disorders (ICHD), the latest version being ICHD-3 (2018). Key clinical characteristics of the headaches, their migraine features and their frequency are key elements in the classification. Within this classification, migraine patients with or without aura (1.1 or 1.2 per ICHD-3) who experience migraines over a specified number of days, or who have chronic migraine (1.3 per ICHD-3) have been evaluated in clinical programs.

There is a need for the improved treatment of headache disorders (especially migraine). Moreover, there is a need for preventive treatments for headache disorders (especially migraine), particularly treatments with different mechanisms of action to provide different treatment options, e.g. in case of non-responsiveness to an existing treatment.

The present invention overcomes one or more of the above-mentioned problems.

### SUMMARY OF THE INVENTION

The present inventors have identified an optimised administration protocol using botulinum neurotoxin A (BoNT/A) that may be effective in the treatment of a headache disorder.

Advantageously, when administered, the BoNT/A may inhibit secretion from a neuron of the central nervous system. The BoNT/A may act upon a neuron of the central nervous system by neuronal (e.g. retrograde) transport.

Additionally, the inventors have identified preferred unit doses, minimum, and maximum doses, together with an optimal injection protocol that may be particularly effective at treating a headache disorder, e.g. by optimally targeting a BoNT/A to suitable nerve endings. In particular, the distribution and number of the injection sites may provide good coverage of BoNT/A to target nerve terminals to inhibit sensory triggering from the trigeminal network, including targets on all three trigeminal branches, and also occipital nerves connected to the spinal sensory trigeminal nucleus.

### DETAILED DESCRIPTION

Thus, in a broad aspect, the invention is directed to a botulinum neurotoxin A (BoNT/A) for use in a method of treating a headache disorder in a subject. The invention also provides, in one aspect, a method of treating a headache disorder in a subject, the method comprising administering a BoNT/A to the subject. In one aspect, the invention provides a use of a BoNT/A in the manufacture of a medicament for treating a headache disorder in a subject.

In one aspect, the invention provides a botulinum neurotoxin A (BoNT/A) for use in a method of treating a headache disorder in a subject, the method comprising administering the BoNT/A to or at an orbicularis oculi muscle and/or a nasalis muscle of the subject.

In a related aspect, the invention provides a method of treating a headache disorder in a subject, the method comprising administering a botulinum neurotoxin A (BoNT/A) to or at an orbicularis oculi muscle and/or a nasalis muscle of the subject.

In a related aspect, the invention provides a use of a botulinum neurotoxin A (BoNT/A) in the manufacture of a medicament for treating a headache disorder in a subject, comprising administering the BoNT/A to or at an orbicularis oculi muscle and/or a nasalis muscle of the subject.

Advantageously, by administering a BoNT/A to or at an orbicularis oculi muscle and/or a nasalis muscle of a subject, the BoNT/A may be targeted to a trigeminal V2 branch (e.g. a maxillary nerve), which branch has been implicated in headache disorders (e.g. migraine).

In one aspect, the invention provides a botulinum neurotoxin A (BoNT/A) for use in a method of treating a headache disorder in a subject by inhibiting secretion from a neuron of the central nervous system.

In a related aspect, the invention provides a method of treating a headache disorder in a subject by inhibiting secretion from a neuron of the central nervous system, the method comprising administering a botulinum neurotoxin A (BoNT/A) to the subject.

In a related aspect, the invention provides a use of a botulinum neurotoxin A (BoNT/A) in the manufacture of a medicament for treating a headache disorder in a subject by inhibiting secretion from a neuron of the central nervous system.

Bacteria in the genus Clostridia produce highly potent and specific protein toxins, which can poison neurons and other cells to which they are delivered. Examples of such clostridial neurotoxins include those produced by C. tetani (TeNT) and by C. botulinum (BoNT) serotypes A-G, and X (see WO 2018/009903 A2), as well as those produced by C. baratii and C. butyricum. Both tetanus and botulinum toxins act by inhibiting the function of affected neurons, specifically the release of neurotransmitters. While botulinum toxin typically acts at the neuromuscular junction and inhibits cholinergic transmission in the peripheral nervous system, tetanus toxin acts in the central nervous system.

In nature, clostridial neurotoxins are synthesised as a single-chain polypeptide that is modified post-translationally by a proteolytic cleavage event to form two polypeptide chains joined together by a disulphide bond. Cleavage occurs at a specific cleavage site, often referred to as the activation site (e.g. activation loop) that is located between the cysteine residues that provide the inter-chain disulphide bond. It is this di-chain form that is the active form of the toxin. The two chains are termed the heavy-chain (H-chain), which has a molecular mass of approximately 100 kDa, and the light-chain (L-chain), which has a molecular mass of approximately 50 kDa. The H-chain comprises an N-terminal translocation component (H_{N} domain) and a C-terminal targeting component (H_{C} domain). The cleavage site is located between the L-chain and the translocation domain components. Following binding of the He domain to its target neuron and internalisation of the bound toxin into the cell via an endosome, the H_{N} domain translocates the L-chain across the endosomal membrane and into the cytosol, and the L-chain provides a protease function (also known as a non-cytotoxic protease).

Non-cytotoxic proteases act by proteolytically cleaving intracellular transport proteins known as SNARE proteins (e.g. SNAP25, VAMP, or Syntaxin). The acronym SNARE derives from the term Soluble NSF Attachment Receptor, where NSF means N-ethylmaleimide-Sensitive Factor. SNARE proteins are integral to intracellular vesicle fusion, and thus to secretion of molecules via vesicle transport from a cell. The protease function is a zinc-dependent endopeptidase activity and exhibits a high substrate specificity for SNARE proteins. Accordingly, once delivered to a desired target cell, the non-cytotoxic protease is capable of inhibiting cellular secretion from the target cell. The L-chain proteases of clostridial neurotoxins are non-cytotoxic proteases that cleave SNARE proteins. In view of the ubiquitous nature of SNARE proteins, clostridial neurotoxins such as botulinum toxin have been successfully employed in a wide range of therapies. For further details on the genetic basis of toxin production in Clostridium botulinum and C. tetani, see Henderson et al (1997) in The Clostridia: Molecular Biology and Pathogenesis, Academic press.

The pharmacological action of a BoNT/A may rely on the cleavage of synaptosomal-associated protein of 25 kDa (SNAP25) within a nerve terminal, thereby inhibiting the fusion and release of vesicle-mediated neurotransmitters.

The term “botulinum neurotoxin A” (BoNT/A) as used herein refers to a polypeptide (e.g. a di-sulphide bonded di-chain polypeptide) comprising a BoNT/A light-chain (L-chain) and a BoNT/A heavy-chain (H-chain). For example, said BoNT/A may consist essentially of (or consist of) a BoNT/A L-chain and a BoNT/A H-chain. Said heavy-chain comprises a BoNT/A translocation domain (H_{N} domain) and a BoNT/A receptor binding domain (H_{C} domain).

The term “consist(s) essentially of” as used in this context means that the BoNT/A does not further comprise one or more amino acid residues that confer additional functionality to the polypeptide, e.g. when administered to a subject. In other words, a polypeptide that “consists essentially of” a BoNT/A L-chain and a BoNT/A H-chain may further comprise one or more amino acid residues (to those of the BoNT/A L-chain and a BoNT/A H-chain) but said one or more further amino acid residues do not confer additional functionality to the polypeptide, e.g. when administered to a subject. Additional functionality may include enzymatic activity, binding activity and/or any physiological activity whatsoever.

A BoNT/A may additionally comprise non-clostridial neurotoxin sequences, e.g. so long as the non-clostridial neurotoxin sequences do not disrupt the ability of the BoNT/A to achieve its therapeutic effect (i.e. to treat a headache disorder). Preferably, the non-clostridial neurotoxin sequence is not one having catalytic activity, e.g. enzymatic activity. In one embodiment a BoNT/A does not comprise a non-clostridial catalytically active domain. In one embodiment, a BoNT/A does not comprise a further catalytically active domain. In one embodiment, the non-clostridial sequence is not one that binds to a cellular receptor. In other words, in one embodiment, the non-clostridial sequence is not a ligand for a cellular receptor. A cellular receptor may be a proteinaceous cellular receptor, such as an integral membrane protein. Examples of cellular receptors can be found in the IUPHAR Guide to Pharmacology Database, version 2019.4, available at https://www.guidetopharmacology.org/download.jsp#db_reports. Non-clostridial neurotoxin sequences may include tags to aid in purification, such as His-tags. In one embodiment, a BoNT/A does not comprise a label or a site for adding a label, such as a sortase acceptor or donor site.

In a preferred embodiment, a BoNT/A does not comprise a therapeutic or diagnostic agent (e.g. a nucleic acid, protein, peptide or small molecule therapeutic or diagnostic agent) additional to the L-chain and H-chain. For example, in one embodiment, a BoNT/A may not comprise a covalently or non-covalently associated therapeutic or diagnostic agent. Thus, a BoNT/A preferably does not function as a delivery vehicle for a further therapeutic or diagnostic agent.

Also encompassed by the term “botulinum neurotoxin A” are variant L-chains and H-chains, preferably wherein said variants have similar binding, translocation, and SNAP25 cleavage properties when compared with a wild-type BoNT/A. More preferably, a variant exhibits improved binding, translocation, and/or SNAP25 cleavage properties when compared with a wild-type BoNT/A. Excluded from the definition “botulinum neurotoxin A” are variants comprising an L-chain (or parts thereof) and/or an H-chain (or parts thereof) derived from a botulinum neurotoxin serotype other than BoNT/A. In other words, chimeric neurotoxins are excluded from the definition “botulinum neurotoxin A”. For example, the term “botulinum neurotoxin A” excludes a polypeptide comprising a BoNT/B He domain (or variant thereof), such as a polypeptide 30 comprising a BoNT/A light-chain, a BoNT/A H_{N} domain, and a BoNT/B He domain (or variant BoNT/B He domain).

A wild-type BoNT/A is one produced by Clostridium botulinum, such as C. botulinum type A, strain ATCC 3502 (the Hall strain). Preferably, a BoNT/A is a wild-type BoNT/A.

A suitable wild-type BoNT/A single-chain polypeptide is provided by SEQ ID NO: 1. A BoNT/A may comprise a polypeptide sequence having at least 70%, 80%, 90%, 95%, 99%, or 99.9% sequence identity to SEQ ID NO: 1 (e.g. when in a single-chain form). Preferably, a BoNT/A may comprise (or consist of) a polypeptide sequence comprising SEQ ID NO: 1 when in a single-chain form. A BoNT/A may comprise (or consist of) SEQ ID NO: 1 when in a single-chain form. Preferably the initial methionine residue is absent.

A BoNT/A for use in the invention is present in a di-chain form, in which the L-chain and H-chain are joined together by a disulphide bond. When produced by and isolated from Clostridium botulinum, the BoNT/A will typically be present in a di-chain form. However, if produced recombinantly (e.g. in E. coli), a single-chain BoNT/A polypeptide (e.g. as described above) obtainable therefrom may be contacted with a protease (e.g. Lys-C) that hydrolyses one or more peptide bond(s) in the activation loop of the BoNT/A, thereby converting the single-chain BoNT/A into a corresponding di-chain BoNT/A. Suitable proteases and methods for cleaving activation loops to produce di-chain clostridial neurotoxins are taught in WO 2014/080206, WO2014/079495, and EP2677029A2, which are incorporated herein by reference. Lys-C may cleave an activation loop C-terminal to one or more of the lysine residues present therein. Where Lys-C cleaves the activation loop more than once, the skilled person will appreciate that a small peptide of the activation loop of a di-chain modified BoNT/A may be absent when compared to a SEQ ID NO shown herein (preferably SEQ ID NO: 5 or 6 may be absent).

The term “obtainable” as used herein also encompasses the term “obtained”.

A di-chain BoNT/A may be a di-chain form of a single-chain BoNT/A, wherein the single-chain BoNT/A comprises a sequence having at least 70%, 80%, 90%, 95%, 99%, or 99.9% sequence identity to SEQ ID NO: 1. Preferably, a di-chain BoNT/A may be a di-chain form of a single-chain BoNT/A, wherein the single-chain BoNT/A comprises (or consists of) SEQ ID NO: 1. Preferably the initial methionine residue is absent.

A di-chain BoNT/A may comprise: (i) an L-chain comprising a polypeptide sequence having at least 70%, 80%, 90%, 95%, 99%, or 99.9% sequence identity to amino acid residues 2-448 of SEQ ID NO: 1; and (ii) an H-chain comprising a polypeptide sequence having at least 70%, 80%, 90%, 95%, 99%, or 99.9% sequence identity to amino acid residues 449-1296 of SEQ ID NO: 1. For example, a di-chain BoNT/A may comprise: (i) an L-chain comprising amino acid residues 2-448 of SEQ ID NO: 1; and (ii) an H-chain comprising amino acid residues 449-1296 of SEQ ID NO: 1. For example, a di-chain BoNT/A may consist of: (i) an L-chain consisting of amino acid residues 2-448 of SEQ ID NO: 1; and (ii) an H-chain consisting of amino acid residues 449-1296 of SEQ ID NO: 1.

A di-chain BoNT/A may comprise: (i) an L-chain comprising a polypeptide sequence having at least 70%, 80%, 90%, 95%, 99%, or 99.9% sequence identity to SEQ ID NO: 2 or 3 (preferably at least 70%, 80%, 90%, 95%, 99%, or 99.9% sequence identity to SEQ ID NO: 2); and (ii) an H-chain comprising a polypeptide sequence having at least 70%, 80%, 90%, 95%, 99%, or 99.9% sequence identity to SEQ ID NO: 4. Preferably, a di-chain BoNT/A may comprise: (i) an L-chain comprising SEQ ID NO: 2 or 3 (preferably comprising SEQ ID NO: 2); and (ii) an H-chain comprising SEQ ID NO: 4. For example, a di-chain BoNT/A may consist of: (i) an L-chain consisting of SEQ ID NO: 2 or 3 (preferably consisting of SEQ ID NO: 2); and (ii) an H-chain consisting of SEQ ID NO: 4. SEQ ID NO: 2 or 3 may comprise a methionine residue at the N-terminus, however, it is preferred that no such N-terminal methionine residue is present.

Thus, a BoNT/A according to the invention comprises an L-chain that is capable of exhibiting non-cytotoxic protease activity and of cleaving SNAP25 in the cytosol of a target neuron. As explained above, the di-chain form is the active form of BoNT/A. Thus, the invention excludes the use of a BoNT/A comprising an L-chain that has been catalytically inactivated (a “catalytically inactive L-chain”), e.g. by way of one or more mutations. Such catalytically inactive L-chains (and clostridial neurotoxins comprising the same) are known in the art. A catalytically inactive L-chain may have one or more mutations that inactivate said catalytic activity. For example, a catalytically inactive L-chain may comprise a mutation of an active site residue. A mutation may be a substitution or a deletion, in particular a substitution with a chemically-similar amino acid. Glutamic acid may be substituted with glutamine, histidine may be substituted with tyrosine, arginine may be substituted with glutamine, and/or tyrosine may be substituted with phenylalanine. Alternatively, any residue may be substituted with alanine. A catalytically inactive BoNT/A L-chain may comprise a mutation at H223, E224, H227, E262, R363, and/or Y366, e.g. a mutation of at least E224 and H227. A catalytically inactive BoNT/A L-chain may comprise a substitution at E224 with glutamine (E224Q) and substitution at H227 with tyrosine (H227Y). The amino acid position numbering may be determined by alignment with SEQ ID NO: 1.

The term “catalytically inactive” as used herein in respect of an L-chain means that said L-chain exhibits substantially no non-cytotoxic protease activity, e.g. no non-cytotoxic protease activity. A catalytically inactive L-chain may be one that does not cleave a protein of the exocytic fusion apparatus in a target cell. The term “substantially no non-cytotoxic protease activity” means that the L-chain has less than 10% or 5% of the non-cytotoxic protease activity of a catalytically active L-chain (preferably an L-chain of native BoNT/A shown as SEQ ID NO: 2 or 3), for example less than 2%, 1% or less than 0.1% of the non-cytotoxic protease activity of a catalytically active L-chain. Non-cytotoxic protease activity can be determined in vitro by incubating a test L-chain with SNAP25 and comparing the amount of SNAP25 cleaved by the test clostridial neurotoxin L-chain when compared to the amount of SNAP25 cleaved by a catalytically active L-chain (preferably an L-chain of native BoNT/A shown as SEQ ID NO: 2 or 3) under the same conditions. Routine techniques, such as SDS-PAGE and Western blotting can be used to quantify the amount of SNAP25 cleaved. Suitable in vitro assays are described in WO 2019/145577 A1, which is incorporated herein by reference.

For the avoidance of doubt, the term “catalytically inactive” as used herein in respect of an L-chain is not intended to encompass an L-chain comprised in a single-chain BoNT/A (the substantially catalytically inactive form of a BoNT/A), wherein, following conversion of the single-chain BoNT/A into a di-chain BoNT/A, the L-chain is capable of exhibiting non-cytotoxic protease activity and of cleaving SNAP25 in the cytosol of a target neuron.

Cell-based and in vivo assays may also be used to determine if a BoNT/A comprising an L-chain and a functional cell binding and translocation domain has non-cytotoxic protease activity. Assays such as the Digit Abduction Score (DAS) assay, the dorsal root ganglia (DRG) assay, spinal cord neuron (SCN) assay, and mouse phrenic nerve hemidiaphragm (PNHD) assay are routine in the art. A suitable assay for determining non-cytotoxic protease activity may be one described in Aoki KR, Toxicon 39: 1815-1820; 2001 or Donald et al (2018), Pharmacol Res Perspect, e00446, 1-14, which are incorporated herein by reference.

The BoNT/A may comprise one or more complexing proteins produced by Clostridium botulinum (e.g. C. botulinum type A, strain ATCC 3502). Said complexing proteins may be haemagglutinin proteins (e.g. ranging between ˜17 and ˜50 kDa in size) and/or non-toxin non-haemagglutinin proteins (e.g. of around 120 kDa in size). Thus, the BoNT/A may be a holotoxin.

It is preferred that a BoNT/A for use in the invention is a BoNT/A isolated from Clostridium botulinum, preferably C. botulinum type A, strain ATCC 3502. More preferably, the BoNT/A is C. botulinum toxin type A haemagglutinin complex (BTX-A-HAC). BTX-A-HAC is the active ingredient present in abobotulinumtoxinA (Dysport®). BTX-A-HAC is a complex comprising (or consisting of) an approximately 150 kilodalton (kDa) polypeptide BoNT/A, a ˜120 kDa non-toxin non-haemagglutinin protein, and various haemagglutinin (HA) proteins ranging between ˜17 and ˜50 kDa in size.

The BoNT/A may be formulated in any suitable manner for administration to a subject, for example as part of a pharmaceutical composition. Such a pharmaceutical composition may comprise a BoNT/A and a pharmaceutically acceptable carrier, excipient, adjuvant, propellant and/or salt. The BoNT/A may be formulated for oral, parenteral, continuous infusion, inhalation or topical application. Compositions suitable for injection may be in the form of solutions, suspensions or emulsions, or dry powders which are dissolved or suspended in a suitable vehicle prior to use. Most preferably, a BoNT/A is formulated for injection, such as intramuscular, intradermal, subdermal, and/or subcutaneous injection, preferably intramuscular injection.

A BoNT/A (preferably BTX-A-HAC) may be present in a pharmaceutical composition further comprising one or more excipients, preferably human serum albumin (e.g. 125 μg human serum albumin US Pharmacopoeia (USP)) and lactose monohydrate (e.g. 2.5 mg lactose USP). A BoNT/A (preferably BTX-A-HAC) may be provided as a lyophilised powder which is reconstituted with 0.9% Sodium Chloride for Injection, USP, prior to use. Most preferably, the pharmaceutical composition (comprising the BoNT/A, most preferably BTX-A-HAC) is abobotulinumtoxinA (Dysport®). AbobotulinumtoxinA (Dysport®) is manufactured by (and can be sourced from) Ipsen Biopharm Limited, Wrexham United Kingdom (UK), including in 300 U and 500 U vials, wherein 1 Unit corresponds to the calculated median lethal intraperitoneal dose (LD50) in mice.

Thus, in the context of a use of a BoNT/A in the manufacture of a medicament for treating a headache disorder described herein, the medicament may be abobotulinumtoxinA (Dysport®).

A treatment according to the invention may comprise administering a BoNT/A to or at one or more of: a frontalis muscle of the subject; a corrugator muscle of the subject; a nasalis muscle of the subject; an orbicularis oculi muscle of the subject; a temporalis muscle of the subject; an occipitalis muscle of the subject; and a trapezius muscle of the subject.

A treatment according to the invention may comprise administering a BoNT/A to 7 of said muscle groups that are implicated in headache (e.g. migraine).

A treatment in accordance with the invention may comprise administering a BoNT/A at at least 7, 8, 9, 10, 11, 12, 13, 14, 15, 16, 17, 18, 19, 20, 21, 22, 23, 24, 25, 26, or 27 sites, preferably at at least 20 or 25 sites in a treatment session. A treatment in accordance with the invention may comprise administering a BoNT/A at up to 35, 30, 28, 26, 24, 22 or 20 sites in a treatment session. A treatment in accordance with the invention may comprise administering a BoNT/A 7-35 sites or 10-30 sites, preferably at 20-30 sites or 25-30 sites in a treatment session. Most preferably, the invention may comprise administering a BoNT/A at 28 sites. Said sites may be injection sites in a treatment session. Preferably, one unit dose of BoNT/A is administered per site in a treatment session. The volume (e.g. of a composition comprising BoNT/A) administered per injection site may be 0.05-0.15 ml, preferably 0.1 ml, per injection site.

Advantageously, the use of at least 20 sites or 25 sites (preferably an optimal 28 sites) may ensure an optimal coverage of the trigeminal network to attenuate the release of one or more mediators (e.g. of nociceptive afferent neurotransmitters, such as CGRP and/or substance P) across all three sensory branches of the trigeminal network and other associated afferent inputs. In particular, administration of unit doses of abobotulinumtoxinA (Dysport®) described herein at 28 injection sites was shown to be particularly effective in the treatment of human subjects with chronic migraine, with a reduction in mean monthly headache days and monthly migraine days observed following treatment.

The administration of a BoNT/A may be to a muscle or at a muscle (preferably, administration to a muscle). Administration to a muscle may be via direct administration to the muscle. Preferably, administration to a muscle is via intramuscular injection. Administration at a muscle may be administration in the region of a muscle, e.g. to the skin overlaying a muscle. In some examples, when administered at a muscle, a BoNT/A may indirectly arrive at the muscle and in other instances the BoNT/A may not arrive at or interact with the muscle. Administration at a muscle may be via intradermal, subdermal, or subcutaneous injection.

A treatment according to the invention may comprise administering a BoNT/A to or at an orbicularis oculi muscle and/or a nasalis muscle of a subject. For example, the treatment may comprise administering a BoNT/A to or at an orbicularis oculi muscle and a nasalis muscle of a subject. Preferably, the invention comprises administering a BoNT/A to or at: a frontalis muscle of the subject; a corrugator muscle of the subject; a nasalis muscle of the subject; an orbicularis oculi muscle of the subject; a temporalis muscle of the subject; an occipitalis muscle of the subject; and a trapezius muscle of the subject.

It is preferred that BoNT/A is administered to or at at least a frontalis muscle of a subject.

When administering a BoNT/A to or at a frontalis muscle, it is preferred that the BoNT/A is administered above the horizontal muscle midline (the midline being defined when a subject is in an upright position). Such administration may be close/closer to the hairline of the subject. For example, the BoNT/A may be administered at one or more sites that are at least 5, 10, 15, 20, 25, 30, or 40 mm above the horizontal frontalis muscle midline.

A trapezius muscle may be an upper trapezius muscle.

Preferably, a trapezius muscle may be an inferior trapezius muscle. Advantageously, administration of a BoNT/A to or at an inferior trapezius muscle may be associated with improved safety and/or reduced side effect(s), e.g. when compared to administration to or at a different trapezius muscle, such as the superior trapezius. For example, administration of a BoNT/A to or at an inferior trapezius muscle may avoid neck (e.g. neck muscle) weakness. For example, administration of a BoNT/A to or at an inferior trapezius muscle may reduce the probability of neck (e.g. neck muscle) weakness.

Administration may comprise administration of a BoNT/A to or at at least a temporalis muscle and/or an occipitalis muscle of a subject.

Preferably, administration in accordance with the invention may exclude administration to or at a procerus muscle, a masseter muscle, and/or a cervical paraspinal muscle. Advantageously, avoiding administration to or at the cervical paraspinal muscles may improve safety and/or reduce one or more side effect(s) associated with BoNT/A treatment. For example, excluding administration to or at the cervical paraspinal muscles may avoid neck (e.g. neck muscle) weakness. For example, excluding administration to or at the cervical paraspinal muscles may reduce the probability of neck (e.g. neck muscle) weakness. For example, administration to or at the procerus may be painful/uncomfortable, therefore, excluding administration to or at the procerus may reduce or avoid pain/discomfort for a subject during treatment.

Preferably, administration in accordance with the invention may exclude administration to or at a semispinalis muscle. Advantageously, avoiding administration to or at a semispinalis muscle may improve safety and/or reduce one or more side effect(s) associated with BoNT/A treatment. For example, excluding administration to or at a semispinalis muscle may avoid neck (e.g. neck muscle) weakness. For example, excluding administration to or at a semispinalis muscle may reduce the probability of neck (e.g. neck muscle) weakness.

1-3 injections of BoNT/A may be administered to or at the frontalis muscle, 1-2 injections of BoNT/A may be administered to or at the corrugator muscle, 1-2 injections of BoNT/A may be administered to or at the nasalis muscle, 1-2 injections of BoNT/A may be administered to or at the orbicularis oculi muscle, 2-5 injections of BoNT/A may be administered to or at the temporalis muscle, 2-5 injections of BoNT/A may be administered to or at the occipitalis muscle, and/or 1-3 injections of BoNT/A may be administered to or at the trapezius muscle.

Administration of a BoNT/A may comprise administering one or more of: 2 injections to or at the frontalis muscle; 1 injection to or at the corrugator muscle; 1 injection to or at the nasalis muscle; 1 injection to or at the orbicularis oculi muscle; 4 injections to or at the temporalis muscle; 3 injections to or at the occipitalis muscle; and 2 injections to or at the trapezius muscle. Preferably, administration of a BoNT/A may comprise: 2 injections to or at a frontalis muscle; 1 injection to or at a corrugator muscle; 1 injection to or at a nasalis muscle; 1 injection to or at an orbicularis oculi muscle; 4 injections to or at a temporalis muscle; 3 injections to or at an occipitalis muscle; and 2 injections to or at a trapezius muscle.

A BoNT/A is preferably administered bilaterally to a subject.

Thus, a treatment according to the invention may comprise administering a BoNT/A to or at: each frontalis muscle of the subject; each corrugator muscle of the subject; each nasalis muscle of the subject; each orbicularis oculi muscle of the subject; each temporalis muscle of the subject; each occipitalis muscle of the subject; and/or each trapezius muscle of the subject. Thus, treatment according to the invention may comprise administering a BoNT/A bilaterally to or at one or more of: each frontalis muscle of the subject; each corrugator muscle of the subject; each nasalis muscle of the subject; each orbicularis oculi muscle of the subject; each temporalis muscle of the subject; each occipitalis muscle of the subject; and each trapezius muscle of the subject. Preferably, the invention comprises administering a BoNT/A to or at: each frontalis muscle of the subject; each corrugator muscle of the subject; each nasalis muscle of the subject; each orbicularis oculi muscle of the subject; each temporalis muscle of the subject; each occipitalis muscle of the subject; and each trapezius muscle of the subject.

More preferably, an administration consists of administering a BoNT/A to or at: each frontalis muscle of the subject; each corrugator muscle of the subject; each nasalis muscle of the subject; each orbicularis oculi muscle of the subject; each temporalis muscle of the subject; each occipitalis muscle of the subject; and each trapezius muscle of the subject. In such instances, administration to or at one or more further muscle(s) is excluded.

1-3 injections of BoNT/A may be administered to or at each frontalis muscle, 1-2 injections of BoNT/A may be administered to or at each corrugator muscle, 1-2 injections of BoNT/A may be administered to or at each nasalis muscle, 1-2 injections of BoNT/A may be administered to or at each orbicularis oculi muscle, 2-5 injections of BoNT/A may be administered to or at each temporalis muscle, 2-5 injections of BoNT/A may be administered to or at each occipitalis muscle, and/or 1-3 injections of BoNT/A may be administered to or at each trapezius muscle.

Administration of a BoNT/A may comprise administering one or more of: 2 injections to or at each frontalis muscle; 1 injection to or at each corrugator muscle; 1 injection to or at each nasalis muscle; 1 injection to or at each orbicularis oculi muscle; 4 injections to or at each temporalis muscle; 3 injections to or at each occipitalis muscle; and 2 injections to or at each trapezius muscle. Preferably, administration of a BoNT/A may comprise administering: 2 injections to or at each frontalis muscle; 1 injection to or at each corrugator muscle; 1 injection to or at each nasalis muscle; 1 injection to or at each orbicularis oculi muscle; 4 injections to or at each temporalis muscle; 3 injections to or at each occipitalis muscle; and 2 injections to or at each trapezius muscle.

More preferably, administration of a BoNT/A may consist of administering: 2 injections to or at each frontalis muscle; 1 injection to or at each corrugator muscle; 1 injection to or at each nasalis muscle; 1 injection to or at each orbicularis oculi muscle; 4 injections to or at each temporalis muscle; 3 injections to or at each occipitalis muscle; and 2 injections to or at each trapezius muscle. In such instances, administration to or at one or more further muscle(s) is excluded.

Yet more preferably, administration of a BoNT/A may comprise administering one or more of: 4 injections to or at the frontalis muscles via 2 injections to or at a frontalis muscle at a first side of the face and 2 injections to or at a frontalis muscle at a second side of the face; 2 injections to or at the corrugator muscles via 1 injection to or at a corrugator muscle at a first side of the face and 1 injection to or at a corrugator muscle at a second side of the face; 2 injections to or at the nasalis muscles via 1 injection to or at a nasalis muscle at a first side of the face and 1 injection to or at a nasalis muscle at a second side of the face; 2 injections to or at the orbicularis oculi muscles via 1 injection to or at an orbicularis oculi muscle at a first side of the face and 1 injection to or at an orbicularis oculi muscle at a second side of the face; 8 injections to or at the temporalis muscles via 4 injections to or at a temporalis muscle at a first side of the head and 4 injections to or at a temporalis muscle at a second side of the head; 6 injections to or at the occipitalis muscles via 3 injections to or at an occipitalis muscle at a first side of the head and 3 injections to or at an occipitalis muscle at a second side of the head; and 4 injections to or at the trapezius muscles via 2 injections to or at a trapezius muscle at a first side of the neck and 2 injections to or at a trapezius muscle at a second side of the neck. Yet more preferably, administration of a BoNT/A may comprise administering: 4 injections to or at the frontalis muscles via 2 injections to or at a frontalis muscle at a first side of the face and 2 injections to or at a frontalis muscle at a second side of the face; 2 injections to or at the corrugator muscles via 1 injection to or at a corrugator muscle at a first side of the face and 1 injection to or at a corrugator muscle at a second side of the face; 2 injections to or at the nasalis muscles via 1 injection to or at a nasalis muscle at a first side of the face and 1 injection to or at a nasalis muscle at a second side of the face; 2 injections to or at the orbicularis oculi muscles via 1 injection to or at an orbicularis oculi muscle at a first side of the face and 1 injection to or at an orbicularis oculi muscle at a second side of the face; 8 injections to or at the temporalis muscles via 4 injections to or at a temporalis muscle at a first side of the head and 4 injections to or at a temporalis muscle at a second side of the head; 6 injections to or at the occipitalis muscles via 3 injections to or at an occipitalis muscle at a first side of the head and 3 injections to or at an occipitalis muscle at a second side of the head; and 4 injections to or at the trapezius muscles via 2 injections to or at a trapezius muscle at a first side of the neck and 2 injections to or at a trapezius muscle at a second side of the neck.

Most preferably, administration of a BoNT/A may consist of administering: 4 injections to or at the frontalis muscles via 2 injections to or at a frontalis muscle at a first side of the face and 2 injections to or at a frontalis muscle at a second side of the face; 2 injections to or at the corrugator muscles via 1 injection to or at a corrugator muscle at a first side of the face and 1 injection to or at a corrugator muscle at a second side of the face; 2 injections to or at the nasalis muscles via 1 injection to or at a nasalis muscle at a first side of the face and 1 injection to or at a nasalis muscle at a second side of the face; 2 injections to or at the orbicularis oculi muscles via 1 injection to or at an orbicularis oculi muscle at a first side of the face and 1 injection to or at an orbicularis oculi muscle at a second side of the face; 8 injections to or at the temporalis muscles via 4 injections to or at a temporalis muscle at a first side of the head and 4 injections to or at a temporalis muscle at a second side of the head; 6 injections to or at the occipitalis muscles via 3 injections to or at an occipitalis muscle at a first side of the head and 3 injections to or at an occipitalis muscle at a second side of the head; and 4 injections to or at the trapezius muscles via 2 injections to or at a trapezius muscle at a first side of the neck and 2 injections to or at a trapezius muscle at a second side of the neck. In such instances, administration to or at one or more further muscle(s) is excluded.

A first and second side (e.g. of the neck or face) may be opposite to one another (e.g. opposite sides). For example, a first side may be a left side (e.g. of the neck or face) and the second side may be right side (e.g. of the neck or face) when looking at a subject.

The number of injections referred to herein preferably refers to the number of injection sites. For example, 4 injections preferably means 4 injection sites.

Advantageously, the above-mentioned administration protocols may be particularly effective when treating a headache disorder, e.g. migraine. The distribution of the trigeminal network may be a primary target for treatment with BoNT/A in accordance with the invention, e.g. those branches with a sensory function. Without wishing to be bound by theory, the afferent sensory input is currently understood to be key for the production of the migraine-triggering neurotransmitters such as CGRP and substance P as well as having up-regulated receptors associated with sensitisation. Where the injection is intramuscular, the muscles in the proximity or which contain these targeted nerve terminals may be injected. The distribution and number of the injection sites may provide good coverage of BoNT/A to target nerve terminals to inhibit sensory triggering from the trigeminal network, e.g. targets on all three branches, and/or also occipital nerves connected to the spinal sensory trigeminal nucleus.

The injection sites are preferably distributed to improve BoNT/A coverage, e.g. to target nerve terminals.

Administration to or at a frontalis muscle and/or a corrugator muscle may target a BoNT/A to a trigeminal V1 branch (e.g. an ophthalmic nerve). Administration to or at a nasalis muscle and/or an orbicularis oculi may target a BoNT/A to a trigeminal V2 branch (e.g. a maxillary nerve). Administration to or at a temporalis muscle may target a BoNT/A to a trigeminal V3 branch (e.g. a mandibular nerve). Administration to or at an occipitalis muscle and/or a trapezius muscle may target a BoNT/A to an occipital nerve (e.g. which may be connected to the spinal sensory trigeminal nucleus).

The term “one or more” as used herein may mean at least 2, 3, 4, 5, 6, 7, 8, 9, 10, 15, or 20. In one embodiment, wherein “one or more” precedes a list, “one or more” may mean all of the members of the list. Similarly, the term “at least one” as used herein may mean at least 2, 3, 4, 5, 6, 7, 8, 9, 10, 15, or 20. In one embodiment, wherein “at least one” precedes a list, “at least one” may mean all of the members of the list.

A BoNT/A may be administered by way of a unit dose. At least 0.25, 0.5, 1, or 2 unit dose(s) may be administered per injection (e.g. per injection site). For example, 0.25, 0.5, 1, or 2 unit dose(s) may be administered per injection (e.g. per injection site). Preferably, 1 unit dose is administered per injection (e.g. per injection site).

When administering a unit dose (or fraction or multiple thereof), this may mean that substantially all of the unit dose (or the fraction or multiple thereof) is administered. For example, a residual amount (e.g. up to 1%, 0.1% or 0.01%) of the unit dose (or the fraction or multiple thereof) may remain in a vial from which the BoNT/A has been taken (e.g. in which the BoNT/A has been reconstituted). For example, a residual amount (e.g. up to 1%, 0.1% or 0.01%) of the unit dose (or the fraction or multiple thereof) may remain in a syringe used to administer the BoNT/A. However, preferably all of the unit dose (or fraction or multiple thereof) is administered (e.g. at one or more injection sites).

Thus, 1-3 unit doses of BoNT/A may be administered to or at the frontalis muscle, 1-2 unit doses of BoNT/A may be administered to or at the corrugator muscle, 1-2 unit doses of BoNT/A may be administered to or at the nasalis muscle, 1-2 unit doses of BoNT/A may be administered to or at the orbicularis oculi muscle, 2-5 unit doses of BoNT/A may be administered to or at the temporalis muscle, 2-5 unit doses of BoNT/A may be administered to or at the occipitalis muscle, and/or 1-3 unit doses of BoNT/A may be administered to or at the trapezius muscle.

A plurality of unit doses may be administered to or at one or more of: a frontalis muscle, a temporalis muscle, an occipitalis muscle, and a trapezius muscle. A single unit dose may be administered to or at one or more of: a corrugator muscle, a nasalis muscle, and an orbicularis oculi muscle. For example, a single unit dose may be administered to or at a corrugator muscle, a nasalis muscle, and an orbicularis oculi muscle, and a plurality of unit doses may be administered to or at a frontalis muscle, a temporalis muscle, an occipitalis muscle, and a trapezius muscle. The plurality of unit doses may be 2-10 unit doses, e.g. 2-8 unit doses, e.g. 2-5 unit doses, such as 2-4 unit doses.

Administration of a BoNT/A may comprise administering one or more of: 2 unit doses to or at the frontalis muscle; 1 unit dose to or at the corrugator muscle; 1 unit dose to or at the nasalis muscle; 1 unit dose to or at the orbicularis oculi muscle; 4 unit doses to or at the temporalis muscle; 3 unit doses to or at the occipitalis muscle; and 2 unit doses to or at the trapezius muscle. Preferably, administration of a BoNT/A may comprise: 2 unit doses to or at the frontalis muscle; 1 unit dose to or at the corrugator muscle; 1 unit dose to or at the nasalis muscle; 1 unit dose to or at the orbicularis oculi muscle; 4 unit doses to or at the temporalis muscle; 3 unit doses to or at the occipitalis muscle; and 2 unit doses to or at the trapezius muscle.

As mentioned, bilateral administration is preferred.

Thus, 1-3 unit doses of BoNT/A may be administered to or at each frontalis muscle, 1-2 unit doses of BoNT/A may be administered to or at each corrugator muscle, 1-2 unit doses of BoNT/A may be administered to or at each nasalis muscle, 1-2 unit doses of BoNT/A may be administered to or at each orbicularis oculi muscle, 2-5 unit doses of BoNT/A may be administered to or at each temporalis muscle, 2-5 unit doses of BoNT/A may be administered to or at each occipitalis muscle, and/or 1-3 unit doses of BoNT/A may be administered to or at each trapezius muscle.

A plurality of unit doses may be administered to or at each frontalis muscle, each temporalis muscle, each occipitalis muscle, and/or each trapezius muscle. A single unit dose may be administered to or at each corrugator muscle, each nasalis muscle, and/or each orbicularis oculi muscle. For example, a single unit dose may be administered to or at each corrugator muscle, each nasalis muscle, and each orbicularis oculi muscle, and a plurality of unit doses may be administered to or at each frontalis muscle, each temporalis muscle, each occipitalis muscle, and each trapezius muscle. The plurality of unit doses may be 2-10 unit doses, e.g. 2-8 unit doses, e.g. 2-5 unit doses, such as 2-4 unit doses.

Administration of a BoNT/A may comprise administering one or more of: 2 unit doses to or at each frontalis muscle; 1 unit dose to or at each corrugator muscle; 1 unit dose to or at each nasalis muscle; 1 unit dose to or at each orbicularis oculi muscle; 4 unit doses to or at each temporalis muscle; 3 unit doses to or at each occipitalis muscle; and 2 unit doses to or at each trapezius muscle.

Preferably, administration of a BoNT/A may comprise administering: 2 unit doses to or at each frontalis muscle; 1 unit dose to or at each corrugator muscle; 1 unit dose to or at each nasalis muscle; 1 unit dose to or at each orbicularis oculi muscle; 4 unit doses to or at each temporalis muscle; 3 unit doses to or at each occipitalis muscle; and 2 unit doses to or at each trapezius muscle.

More preferably, administration of a BoNT/A may consist of administering: 2 unit doses to or at each frontalis muscle; 1 unit dose to or at each corrugator muscle; 1 unit dose to or at each nasalis muscle; 1 unit dose to or at each orbicularis oculi muscle; 4 unit doses to or at each temporalis muscle; 3 unit doses to or at each occipitalis muscle; and 2 unit doses to or at each trapezius muscle. In such instances, administration to or at one or more further muscle(s) is excluded.

Yet more preferably, administration of a BoNT/A may comprise administering one or more of: 4 unit doses to or at the frontalis muscles via 2 unit doses to or at a frontalis muscle at a first side of the face of the subject and 2 unit doses to or at a frontalis muscle at a second side of the face of the subject; 2 unit doses to or at the corrugator muscles via 1 unit dose to or at a corrugator muscle at a first side of the face of the subject and 1 unit dose to or at a corrugator muscle at a second side of the face of the subject; 2 unit doses to or at the nasalis muscles via 1 unit dose to or at a nasalis muscle at a first side of the face of the subject and 1 injection to or at a nasalis muscle at a second side of the face of the subject; 2 unit doses to or at the orbicularis oculi muscles via 1 unit dose to or at an orbicularis oculi muscle at a first side of the face of the subject and 1 unit dose to or at an orbicularis oculi muscle at a second side of the face of the subject; 8 unit doses to or at the temporalis muscles via 4 unit doses to or at a temporalis muscle at a first side of the head of the subject and 4 unit doses to or at a temporalis muscle at a second side of the head of the subject; 6 unit doses to or at the occipitalis muscles via 3 unit doses to or at an occipitalis muscle at a first side of the head of the subject and 3 unit doses to or at an occipitalis muscle at a second side of the head of the subject; and 4 unit doses to or at the trapezius muscles via 2 unit doses to or at a trapezius muscle at a first side of the neck of the subject and 2 unit doses to or at a trapezius muscle at a second side of the neck of the subject. Yet more preferably, administration of a BoNT/A may comprise administering: 4 unit doses to or at the frontalis muscles via 2 unit doses to or at a frontalis muscle at a first side of the face of the subject and 2 unit doses to or at a frontalis muscle at a second side of the face of the subject; 2 unit doses to or at the corrugator muscles via 1 unit dose to or at a corrugator muscle at a first side of the face of the subject and 1 unit dose to or at a corrugator muscle at a second side of the face of the subject; 2 unit doses to or at the nasalis muscles via 1 unit dose to or at a nasalis muscle at a first side of the face of the subject and 1 injection to or at a nasalis muscle at a second side of the face of the subject; 2 unit doses to or at the orbicularis oculi muscles via 1 unit dose to or at an orbicularis oculi muscle at a first side of the face of the subject and 1 unit dose to or at an orbicularis oculi muscle at a second side of the face of the subject; 8 unit doses to or at the temporalis muscles via 4 unit doses to or at a temporalis muscle at a first side of the head of the subject and 4 unit doses to or at a temporalis muscle at a second side of the head of the subject; 6 unit doses to or at the occipitalis muscles via 3 unit doses to or at an occipitalis muscle at a first side of the head of the subject and 3 unit doses to or at an occipitalis muscle at a second side of the head of the subject; and 4 unit doses to or at the trapezius muscles via 2 unit doses to or at a trapezius muscle at a first side of the neck of the subject and 2 unit doses to or at a trapezius muscle at a second side of the neck of the subject.

Most preferably, administration of a BoNT/A may consist of administering: 4 unit doses to or at the frontalis muscles via 2 unit doses to or at a frontalis muscle at a first side of the face of the subject and 2 unit doses to or at a frontalis muscle at a second side of the face of the subject; 2 unit doses to or at the corrugator muscles via 1 unit dose to or at a corrugator muscle at a first side of the face of the subject and 1 unit dose to or at a corrugator muscle at a second side of the face of the subject; 2 unit doses to or at the nasalis muscles via 1 unit dose to or at a nasalis muscle at a first side of the face of the subject and 1 injection to or at a nasalis muscle at a second side of the face of the subject; 2 unit doses to or at the orbicularis oculi muscles via 1 unit dose to or at an orbicularis oculi muscle at a first side of the face of the subject and 1 unit dose to or at an orbicularis oculi muscle at a second side of the face of the subject; 8 unit doses to or at the temporalis muscles via 4 unit doses to or at a temporalis muscle at a first side of the head of the subject and 4 unit doses to or at a temporalis muscle at a second side of the head of the subject; 6 unit doses to or at the occipitalis muscles via 3 unit doses to or at an occipitalis muscle at a first side of the head of the subject and 3 unit doses to or at an occipitalis muscle at a second side of the head of the subject; and 4 unit doses to or at the trapezius muscles via 2 unit doses to or at a trapezius muscle at a first side of the neck of the subject and 2 unit doses to or at a trapezius muscle at a second side of the neck of the subject. In such instances, administration to or at one or more further muscle(s) is excluded.

In one embodiment, administration of a BoNT/A according to the invention may exclude administering to any further muscle(s) than the muscle(s) indicated. In other words, in one embodiment, administration of a BoNT/A may be to the muscle(s) indicated only.

The total number of unit doses administered in a treatment session may be at least 7, 8, 9, 10, 11, 12, 13, 14, 15, 16, 17, 18, 19, 20, 21, 22, 23, 24, 25, 26, or 27 unit doses, preferably at least 20 or 25 unit doses. The total number of unit doses administered in a treatment session may be up to 35, 30, 28, 26, 24, 22 or 20 unit doses. The total number of unit doses administered in a treatment session may be 7-35 unit doses or 10-30 unit doses, preferably 20-30 unit doses or 25-30 unit doses.

Most preferably, 28 unit doses are administered in a treatment session.

A unit dose may be quantified using LD50 Units. LD50 is preferably determined using a mouse LD50 assay according to standard techniques. In said assay, 1 Unit (U) is defined as an amount of the BoNT/A that corresponds to the calculated median lethal dose (LD50) in mice. Preferably, the calculated median lethal intraperitoneal dose in mice.

A unit dose (1 unit dose) may be 4-20 U. The lower end of said range may be: 5, 6, 7, 8, 9, 10, 11, 12, 13, 14, 15, 16, 17, 18, or 19 U. Preferably, the lower end of said range is 6. The upper end of said range may be: 19, 18, 17, 16, 15, 14, 13, 12, 11, 10, 9, 8, 7, 6, or 5 U.

A unit dose may be 6-9.5 U.

Preferably, a unit dose may be 4-14 U, more preferably 8-10 U, even more preferably 9.09 U (e.g. rounded to 9 U).

A unit dose may be 11-20 U.

Yet more preferably, a unit dose may be 10-20 U, more preferably 14-16 U, most preferably 15.15 U (e.g. rounded to 15 U).

The unit dose (1 unit dose) administered to or at each muscle may be the same unit dose, e.g. 9.09 U or 15 U. The total number of unit doses administered to or at each muscle may be different.

A total dose administered in a treatment session may be up to: 560, 550, 540, 530, 520, 510, 500, 490, 480, 470, 460, 450, 448, 440, 430, 424, 420, 410, 400, 392, 390, 380, 370, 360, 350, 340, 330, 320, 310, 300, 290, 280, 270, 260, 255, 250, 240, 230, 220, 210, 200, 190, 180, 170, 160, 150, 140, 130, or 120 U. Preferably, the total dose in a treatment session may be up to 500 U, more preferably up to 255 U, even more preferably up to 424 U.

The term “up to” when used in reference to a value (e.g. up to 560 U) means up to and including the value recited.

A total dose administered in a treatment session may be at least: 110, 112, 120, 130, 140, 150, 160, 170, 180, 190, 200, 210, 220, 224, 230, 240, 250, 260, 270, 280, 290, 300, 310, 320, 330, 340, 350, 360, 370, 380, 390, 392, 400, 410, 419, 420, 430, 440, 450, 460, 470, 480, 490, 500, 510, 520, 530, or 540 U. Preferably, the total dose in a treatment session may be at least 250 U. More preferably, a total dose administered in a treatment session may be at least 300 U or 310 U, such as at least 419 U.

Advantageously, a total dose of at least 300 U or 310 U (such as at least 419 U) may be particularly effective at treating a headache disorder. This may be particularly effective when administration comprises administering:
(a) 2 unit doses of BoNT/A to or at each frontalis muscle of the subject;
(b) 1 unit dose of BoNT/A to or at each corrugator muscle of the subject;
(c) 1 unit dose of BoNT/A to or at each nasalis muscle of the subject;
(d) 1 unit dose of BoNT/A to or at each orbicularis oculi muscle of the subject;
(e) 4 unit doses of BoNT/A to or at each temporalis muscle of the subject;
(f) 3 unit doses of BoNT/A to or at each occipitalis muscle of the subject; and
(g) 2 unit doses of BoNT/A to or at each trapezius muscle of the subject, wherein 1 unit dose is administered per injection site.

A total dose administered in a treatment session may be 112-560 U. The lower end of said range may be: 120, 130, 140, 150, 160, 170, 180, 190, 200, 210, 220, 224, 230, 240, 250, 260, 270, 280, 290, 300, 310, 320, 330, 340, 350, 360, 370, 380, 390, 392, 400, 410, 419, 420, 430, 440, 450, 460, 470, 480, 490, 500, 510, 520, 530, or 540 U. The upper end of said range may be: 550, 540, 530, 520, 510, 500, 490, 480, 470, 460, 450, 448, 440, 430, 424, 420, 410, 400, 392, 390, 380, 370, 360, 350, 340, 330, 320, 310, 300, 290, 280, 270, 260, 255, 250, 240, 230, 220, 210, 200, 190, 180, 170, 160, 150, 140, 130, or 120 U. A total dose administered during treatment may be 112-560 U, 280-560 U, 112-448 U, 392-448 U, 112-392 U, or 224-280 U. A total dose administered in a treatment session may be 224-280 U (e.g. 255 U). Preferably, a total dose administered in a treatment session may be 300-448 U, 310-448 U, or 392-448 U. Most preferably, the total dose administered in a treatment session may be 424 U.

Preferably, a unit dose is 8-10 U and the total dose is up to 280 U (e.g. 224-280 U). More preferably, a unit dose is 9.09 U (e.g. rounded to 9 U) and the total dose is up to 255 U (e.g. is 255 U).

Yet more preferably, a unit dose is 14-16 U and the total dose is up to 448 U (e.g. 392-448 U). More preferably, a unit dose is 15.15 U (e.g. rounded to 15 U) and the total dose is up to 424 U (e.g. is 424 U).

Advantageously, said unit doses (e.g. 9.09 U and 15.15 U) correspond to a dose per injection site believed to maximise the potential for demonstrating efficacy while remaining within doses per site/muscle where a positive pharmacologic effect has been observed consistently for abobotulinumtoxinA (Dysport®). The total doses per muscle described herein is within or below the range of doses of abobotulinumtoxinA (Dysport®) shown to be associated with an acceptable safety profile. An advantage associated with the total doses per muscle is that when abobotulinumtoxinA (Dysport®) is employed, from a practical perspective, a single vial can be used for either the 424 U or 255 U total dose via reconstitution within a 500 U vial for the higher dose or 300 U vial for the lower dose, with 3.3 ml of saline added to the vial for reconstitution and 2.8 ml readily extracted from the vial for injection, e.g. of 0.1 ml to 28 sites as preferred herein—this makes administration much easier for a physician.

The above-referenced unit doses and/or total doses may be particularly relevant when the BoNT/A is BTX-A-HAC, preferably wherein the BTX-A-HAC is part of a pharmaceutical composition, most preferably wherein the pharmaceutical composition (comprising the BoNT/A, most preferably BTX-A-HAC) is abobotulinumtoxinA (Dysport®). In other words, the pharmaceutical composition, including the BoNT/A (most preferably BTX-A-HAC), is preferably abobotulinumtoxinA (Dysport®). Thus, preferably, a Unit (LD50 Unit) described herein may be an abobotulinumtoxinA (Dysport®) Unit (LD50 Unit).

Thus, in one highly preferred aspect, the invention provides BTX-A-HAC (preferably abobotulinumtoxinA (Dysport®)) for use in a method of treating a headache disorder in a subject, the method comprising administering:
(a) 2 unit doses of BTX-A-HAC (preferably abobotulinumtoxinA (Dysport®)) to or at each frontalis muscle of the subject;
(b) 1 unit dose of BTX-A-HAC (preferably abobotulinumtoxinA (Dysport®)) to or at each corrugator muscle of the subject;
(c) 1 unit dose of BTX-A-HAC (preferably abobotulinumtoxinA (Dysport®)) to or at each nasalis muscle of the subject;
(d) 1 unit dose of BTX-A-HAC (preferably abobotulinumtoxinA (Dysport®)) to or at each orbicularis oculi muscle of the subject;
(e) 4 unit doses of BTX-A-HAC (preferably abobotulinumtoxinA (Dysport®)) to or at each temporalis muscle of the subject;
(f) 3 unit doses of BTX-A-HAC (preferably abobotulinumtoxinA (Dysport®)) to or at each occipitalis muscle of the subject; and
(g) 2 unit doses of BTX-A-HAC (preferably abobotulinumtoxinA (Dysport®)) to or at each trapezius muscle of the subject, and
wherein 1 unit dose is 4-20 Units (U) of BTX-A-HAC (preferably abobotulinumtoxinA (Dysport®)), wherein 1 U corresponds to the calculated median lethal intraperitoneal dose (LD50) in mice, and wherein the total dose administered during treatment (in a treatment session) is up to 560 U (e.g. is 112-560 U) of BTX-A-HAC (preferably abobotulinumtoxinA (Dysport®)).

In a related highly preferred aspect, the invention provides a method of treating a headache disorder in a subject, the method comprising administering:
(a) 2 unit doses of BTX-A-HAC (preferably abobotulinumtoxinA (Dysport®)) to or at each frontalis muscle of the subject;
(b) 1 unit dose of BTX-A-HAC (preferably abobotulinumtoxinA (Dysport®)) to or at each corrugator muscle of the subject;
(c) 1 unit dose of BTX-A-HAC (preferably abobotulinumtoxinA (Dysport®)) to or at each nasalis muscle of the subject;
(d) 1 unit dose of BTX-A-HAC (preferably abobotulinumtoxinA (Dysport®)) to or at each orbicularis oculi muscle of the subject;
(e) 4 unit doses of BTX-A-HAC (preferably abobotulinumtoxinA (Dysport®)) to or at each temporalis muscle of the subject;
(f) 3 unit doses of BTX-A-HAC (preferably abobotulinumtoxinA (Dysport®)) to or at each occipitalis muscle of the subject; and
(g) 2 unit doses of BTX-A-HAC (preferably abobotulinumtoxinA (Dysport®)) to or at each trapezius muscle of the subject, and
wherein 1 unit dose is 4-20 Units (U) of BTX-A-HAC (preferably abobotulinumtoxinA (Dysport®)), wherein 1 U corresponds to the calculated median lethal intraperitoneal dose (LD50) in mice, and wherein the total dose administered during treatment (in a treatment session) is up to 560 U (e.g. is 112-560 U, preferably 300-448 U) of BTX-A-HAC (preferably abobotulinumtoxinA (Dysport®)).

In a related highly preferred aspect, the invention provides a use of BTX-A-HAC (preferably abobotulinumtoxinA (Dysport®)) in the manufacture of a medicament for treating a headache disorder in a subject, comprising administering:
(a) 2 unit doses of BTX-A-HAC (preferably abobotulinumtoxinA (Dysport®)) to or at each frontalis muscle of the subject;
(b) 1 unit dose of BTX-A-HAC (preferably abobotulinumtoxinA (Dysport®)) to or at each corrugator muscle of the subject;
(c) 1 unit dose of BTX-A-HAC (preferably abobotulinumtoxinA (Dysport®)) to or at each nasalis muscle of the subject;
(d) 1 unit dose of BTX-A-HAC (preferably abobotulinumtoxinA (Dysport®)) to or at each orbicularis oculi muscle of the subject;
(e) 4 unit doses of BTX-A-HAC (preferably abobotulinumtoxinA (Dysport®)) to or at each temporalis muscle of the subject;
(f) 3 unit doses of BTX-A-HAC (preferably abobotulinumtoxinA (Dysport®)) to or at each occipitalis muscle of the subject; and
(g) 2 unit doses of BTX-A-HAC (preferably abobotulinumtoxinA (Dysport®)) to or at each trapezius muscle of the subject, and
wherein 1 unit dose is 4-20 Units (U) of BTX-A-HAC (preferably abobotulinumtoxinA (Dysport®)), wherein 1 U corresponds to the calculated median lethal intraperitoneal dose (LD50) in mice, and wherein the total dose administered during treatment (in a treatment session) is up to 560 U (e.g. is 112-560 U, preferably 300-448 U) of BTX-A-HAC (preferably abobotulinumtoxinA (Dysport®)).

Thus, in one highly preferred aspect, the invention provides abobotulinumtoxinA (Dysport®) for use in a method of treating a headache disorder in a subject, the method comprising administering:
(a) 2 unit doses of abobotulinumtoxinA (Dysport®) to or at each frontalis muscle of the subject;
(b) 1 unit dose of abobotulinumtoxinA (Dysport®) to or at each corrugator muscle of the subject;
(c) 1 unit dose of abobotulinumtoxinA (Dysport®) to or at each nasalis muscle of the subject;
(d) 1 unit dose of abobotulinumtoxinA (Dysport®) to or at each orbicularis oculi muscle of the subject;
(e) 4 unit doses of abobotulinumtoxinA (Dysport®) to or at each temporalis muscle of the subject;
(f) 3 unit doses of abobotulinumtoxinA (Dysport®) to or at each occipitalis muscle of the subject; and
(g) 2 unit doses of abobotulinumtoxinA (Dysport®) to or at each trapezius muscle of the subject, and
wherein 1 unit dose is 4-20 Units (U) of abobotulinumtoxinA (Dysport®), wherein 1 U corresponds to the calculated median lethal intraperitoneal dose (LD50) in mice, and wherein the total dose administered during treatment (in a treatment session) is up to 560 U (e.g. is 112-560 U) of abobotulinumtoxinA (Dysport®).

In a related highly preferred aspect, the invention provides a method of treating a headache disorder in a subject, the method comprising administering:
(a) 2 unit doses of abobotulinumtoxinA (Dysport®) to or at each frontalis muscle of the subject;
(b) 1 unit dose of abobotulinumtoxinA (Dysport®) to or at each corrugator muscle of the subject;
(c) 1 unit dose of abobotulinumtoxinA (Dysport®) to or at each nasalis muscle of the subject;
(d) 1 unit dose of abobotulinumtoxinA (Dysport®) to or at each orbicularis oculi muscle of the subject;
(e) 4 unit doses of abobotulinumtoxinA (Dysport®) to or at each temporalis muscle of the subject;
(f) 3 unit doses of abobotulinumtoxinA (Dysport®) to or at each occipitalis muscle of the subject; and
(g) 2 unit doses of abobotulinumtoxinA (Dysport®) to or at each trapezius muscle of the subject, and
wherein 1 unit dose is 4-20 Units (U) of abobotulinumtoxinA (Dysport®), wherein 1 U corresponds to the calculated median lethal intraperitoneal dose (LD50) in mice, and wherein the total dose administered during treatment (in a treatment session) is up to 560 U (e.g. is 112-560 U, preferably 300-448 U) of abobotulinumtoxinA (Dysport®).

In a related highly preferred aspect, the invention provides a use of abobotulinumtoxinA (Dysport®) in the manufacture of a medicament for treating a headache disorder in a subject, comprising administering:
(a) 2 unit doses of abobotulinumtoxinA (Dysport®) to or at each frontalis muscle of the subject;
(b) 1 unit dose of abobotulinumtoxinA (Dysport®) to or at each corrugator muscle of the subject;
(c) 1 unit dose of abobotulinumtoxinA (Dysport®) to or at each nasalis muscle of the subject;
(d) 1 unit dose of abobotulinumtoxinA (Dysport®) to or at each orbicularis oculi muscle of the subject;
(e) 4 unit doses of abobotulinumtoxinA (Dysport®) to or at each temporalis muscle of the subject;
(f) 3 unit doses of abobotulinumtoxinA (Dysport®) to or at each occipitalis muscle of the subject; and
(g) 2 unit doses of abobotulinumtoxinA (Dysport®) to or at each trapezius muscle of the subject, and
wherein 1 unit dose is 4-20 Units (U) of abobotulinumtoxinA (Dysport®), wherein 1 U corresponds to the calculated median lethal intraperitoneal dose (LD50) in mice, and wherein the total dose administered during treatment (in a treatment session) is up to 560 U (e.g. is 112-560 U, preferably 300-448 U) of abobotulinumtoxinA (Dysport®).

Administration of BoNT/A may comprise (more preferably consist of):
(a) 4 intramuscular injections of abobotulinumtoxinA (Dysport®) to the frontalis muscles via 2 intramuscular injections of abobotulinumtoxinA (Dysport®) to a frontalis muscle at a first side of the face and 2 intramuscular injections of abobotulinumtoxinA (Dysport®) to a frontalis muscle at a second side of the face;
(b) 2 intramuscular injections of abobotulinumtoxinA (Dysport®) to the corrugator muscles via 1 intramuscular injection of abobotulinumtoxinA (Dysport®) to a corrugator muscle at a first side of the face and 1 intramuscular injection of abobotulinumtoxinA (Dysport®) to a corrugator muscle at a second side of the face;
(c) 2 intramuscular injections of abobotulinumtoxinA (Dysport®) to the nasalis muscles via 1 intramuscular injection of abobotulinumtoxinA (Dysport®) to a nasalis muscle at a first side of the face and 1 intramuscular injection of abobotulinumtoxinA (Dysport®) to a nasalis muscle at a second side of the face;
(d) 2 intramuscular injections of abobotulinumtoxinA (Dysport®) to the orbicularis oculi muscles via 1 injection of abobotulinumtoxinA (Dysport®) to an orbicularis oculi muscle at a first side of the face and 1 intramuscular injection of abobotulinumtoxinA (Dysport®) to an orbicularis oculi muscle at a second side of the face;
(e) 8 intramuscular injections of abobotulinumtoxinA (Dysport®) to the temporalis muscles via 4 intramuscular injections of abobotulinumtoxinA (Dysport®) to a temporalis muscle at a first side of the head and 4 intramuscular injections of abobotulinumtoxinA (Dysport®) to a temporalis muscle at a second side of the head;
(f) 6 intramuscular injections of abobotulinumtoxinA (Dysport®) to the occipitalis muscles via 3 intramuscular injections of abobotulinumtoxinA (Dysport®) to an occipitalis muscle at a first side of the head and 3 intramuscular injections of abobotulinumtoxinA (Dysport®) to an occipitalis muscle at a second side of the head; and
(g) 4 intramuscular injections of abobotulinumtoxinA (Dysport®) to the trapezius muscles via 2 intramuscular injections of abobotulinumtoxinA (Dysport®) to a trapezius muscle at a first side of the neck and 2 intramuscular injections of abobotulinumtoxinA (Dysport®) to a trapezius muscle at a second side of the neck; and
wherein 9 U (e.g. 9.09 U) of abobotulinumtoxinA (Dysport®) is administered per injection. 10 Administration of BoNT/A may comprise (more preferably consist of):
(h) 4 intramuscular injections of abobotulinumtoxinA (Dysport®) to the frontalis muscles via 2 intramuscular injections of abobotulinumtoxinA (Dysport®) to a frontalis muscle at a first side of the face and 2 intramuscular injections of abobotulinumtoxinA (Dysport®) to a frontalis muscle at a second side of the face;
(i) 2 intramuscular injections of abobotulinumtoxinA (Dysport®) to the corrugator muscles via 1 intramuscular injection of abobotulinumtoxinA (Dysport®) to a corrugator muscle at a first side of the face and 1 intramuscular injection of abobotulinumtoxinA (Dysport®) to a corrugator muscle at a second side of the face;
(j) 2 intramuscular injections of abobotulinumtoxinA (Dysport®) to the nasalis muscles via 1 intramuscular injection of abobotulinumtoxinA (Dysport®) to a nasalis muscle at a first side of the face and 1 intramuscular injection of abobotulinumtoxinA (Dysport®) to a nasalis muscle at a second side of the face;
(k) 2 intramuscular injections of abobotulinumtoxinA (Dysport®) to the orbicularis oculi muscles via 1 injection of abobotulinumtoxinA (Dysport®) to an orbicularis oculi muscle at a first side of the face and 1 intramuscular injection of abobotulinumtoxinA (Dysport®) to an orbicularis oculi muscle at a second side of the face;
(l) 8 intramuscular injections of abobotulinumtoxinA (Dysport®) to the temporalis muscles via 4 intramuscular injections of abobotulinumtoxinA (Dysport®) to a temporalis muscle at a first side of the head and 4 intramuscular injections of abobotulinumtoxinA (Dysport®) to a temporalis muscle at a second side of the head;
(m)6 intramuscular injections of abobotulinumtoxinA (Dysport®) to the occipitalis muscles via 3 intramuscular injections of abobotulinumtoxinA (Dysport®) to an occipitalis muscle at a first side of the head and 3 intramuscular injections of abobotulinumtoxinA (Dysport®) to an occipitalis muscle at a second side of the head; and
(n) 4 intramuscular injections of abobotulinumtoxinA (Dysport®) to the trapezius muscles via 2 intramuscular injections of abobotulinumtoxinA (Dysport®) to a trapezius muscle at a first side of the neck and 2 intramuscular injections of abobotulinumtoxinA (Dysport®) to a trapezius muscle at a second side of the neck; and
   wherein 15 U (e.g. 15.15 U) of abobotulinumtoxinA (Dysport®) is administered per injection.

A headache disorder may be a: muscular/myogenic headache, tension headache, vascular headache (e.g. migraine), high blood pressure headache, traction and inflammatory headache (which are usually symptoms of other disorders, ranging from stroke to sinus infection), hormone headache, rebound headache, chronic sinusitis headache, organic headache, and/or ictal headache (associated with seizure activity).

Muscular/myogenic headaches may involve the tightening or tensing of facial and neck muscles; they may radiate to the forehead. Tension headache is the most common form of myogenic headache. A tension headache is a condition involving pain or discomfort in the head, scalp, or neck, usually associated with muscle tightness in these areas. Tension headaches typically result from the contraction of neck and scalp muscles. One cause of this muscle contraction may be a response to stress, depression or anxiety. Any activity that causes the head to be held in one position for a long time without moving may be a cause of headache. Such activities include typing or use of computers, fine work with the hands, and use of a microscope. Sleeping in a cold room or sleeping with the neck in an abnormal position may also trigger this type of headache. A tension-type headache, includes, without limitation, an episodic tension headache and a chronic tension headache.

The most common type of vascular headache is migraine. Other kinds of vascular headaches may include cluster headaches, which cause repeated episodes of intense pain, and headaches resulting from high blood pressure.

A migraine is a heterogeneous disorder that generally involves recurring headaches. Migraines may be different from other headaches as they may occur with other symptoms, such as, e.g., nausea, vomiting, or sensitivity to light. For example, in some instances, a throbbing pain may be felt only on one side of the head. Clinical features such as type of aura symptoms, presence of prodromes, or associated symptoms such as vertigo, may be seen in subgroups of subjects with different underlying pathophysiological and genetic mechanisms. A migraine may be, without limitation, a migraine without aura (common migraine), a migraine with aura (classic migraine), a menstrual migraine, a migraine equivalent (acephalic headache), a probable migraine, a complicated migraine, an abdominal migraine and/or a mixed tension migraine.

Preferably, a headache disorder is migraine. Thus, preferably, the invention is directed to the treatment of migraine.

The migraine may be episodic migraine or chronic migraine (preferably chronic migraine). A subject may have episodic migraine if the subject experiences headaches (e.g. migraine) on fewer than 15 days per month (e.g. at least 1 but less than 15 days per month), preferably if the subject experiences headaches (e.g. migraine) on at least 4 but less than 15 days per month. In other words, episodic migraine may be defined as headache (e.g. migraine) on fewer than 15 days per month (e.g. at least 1 but less than 15 days per month), preferably as headache (e.g. migraine) on at least 4 but less than 15 days per month. In addition to headache pain, a migraine may be associated with one or more additional symptom(s), including increased light sensitivity, nausea, and/or vomiting.

A headache disorder may be classified based on the diagnostic criteria and definitions provided in The International Classification of Headache Disorders 3rd edition (2018) (ICHD-3).

A migraine without aura may be diagnosed based on the following ICHD-3 criteria:
A. At least five attacks fulfilling criteria B-D
B. Headache attacks lasting 4-72 hours (when untreated or unsuccessfully treated)
C. Headache has at least two of the following four characteristics:
   unilateral location
   pulsating quality
   moderate or severe pain intensity
   aggravation by or causing avoidance of routine physical activity (e.g. walking or climbing stairs)
D. During headache at least one of the following:
   nausea and/or vomiting
   photophobia and phonophobia
E. Not better accounted for by another ICHD-3 diagnosis.

A migraine with aura may be diagnosed based on the following ICHD-3 criteria:
A. At least two attacks fulfilling criteria B and C
B. One or more of the following fully reversible aura symptoms:
   Visual
   Sensory
   speech and/or language
   motor
   brainstem
   retinal
C. At least three of the following six characteristics:
   at least one aura symptom spreads gradually over ≥5 minutes
   two or more aura symptoms occur in succession
   each individual aura symptom lasts 5-60 minutes
   at least one aura symptom is unilateral
   at least one aura symptom is positive
   the aura is accompanied, or followed within 60 minutes, by headache
D. Not better accounted for by another ICHD-3 diagnosis.

A chronic migraine may be diagnosed based on the following ICHD-3 criteria:
A. Headache (migraine-like or tension-type-like) on ≥15 days/month for >3 months, and fulfilling criteria B and C
B. Occurring in a patient who has had at least five attacks fulfilling criteria B-D for “migraine without aura” (see above) and/or criteria B and C for “migraine with aura” (see above)
C. On ≥8 days/month for >3 months, fulfilling any of the following:
   1 criteria C and D for “migraine without aura” (see above)
   2 criteria B and C for “migraine with aura” (see above) believed by the patient to be migraine at onset and relieved by a triptan or ergot derivative
D. Not better accounted for by another ICHD-3 diagnosis.

Without wishing to be bound by theory, the pathophysiology of migraine attacks, whether episodic or chronic, is believed to include alteration of brain excitability, intracranial arterial dilatation and the recurrent activation and sensitization of the trigemino-vascular pathways. Activation of the trigeminal nerves is believed to trigger the release of mediators (e.g. neuropeptides) including CGRP, neurokinin A (formerly known as substance K) and substance P. Not only may these be vasoactive (CGRP is widely accepted to be important in localized vasodilation in capillary beds), they may also be important in nociceptive processing. Furthermore, these mediators (e.g. neuropeptides) are believed to promote the release of inflammatory mediators (e.g. tumour necrosis factor-alpha) which further stimulates CGRP production. All the aforementioned mediators (e.g. neurotransmitters) may be released from nerve terminals through the docking and fusion of neurotransmitter-containing vesicles through the SNARE complex. The SNARE complex is a highly conserved series of proteins, all of which need to be intact for the successful docking and fusion of vesicles to the nerve terminal membrane. One of these proteins, SNAP25, is cleaved by BoNT/A once the active component of the toxin has entered the nerve terminal, which may result in an inhibition of vesicle mediated mediator (e.g. neurotransmitter) release from trigeminal nerve terminals. The release of CGRP is believed to be involved in the triggering of migraines and the successful prevention of migraines through the use of CGRP antagonists attests to the importance of preventing the action of CGRP. However, the release of other mediators (e.g. nociceptive stimulants) such as substance P may also be inhibited by BoNT/A. Also, since the same vesicles containing neurotransmitters may be used as a mechanism for shuttling and inserting receptors into the nerve terminal, the disruption of vesicle fusion with BoNT/A may also reduce the density of receptors important for pain signal transduction (e.g. TRPV1, P2X3) which can become upregulated in headache (e.g. migraine) sufferers and may contribute to peripheral/central sensitization. The down-regulation of such nociceptive receptors may further attenuate the sensory triggering of a headache disorder (e.g. migraine). Thus, there are multiple targets following administration of BoNT/A that may result in an overall reduction in the afferent input from the trigeminal nerves triggering a headache disorder (e.g. migraines).

Cluster headaches affect one side of the head (are unilateral) and may be associated with tearing of the eyes and nasal congestion. They usually occur in clusters, happening repeatedly every day at the same time for several weeks and then remitting.

Rebound headaches, also known as medication overuse headaches, occur when medication is taken too frequently to relieve a headache. Rebound headaches frequently occur daily and can be very painful.

Sinusitis is inflammation, either bacterial, fungal, viral, allergic or autoimmune, of the paranasal sinuses. Chronic sinusitis is one of the most common complications of the common cold.

Symptoms may include: nasal congestion; facial pain; headache; fever; general malaise; thick green or yellow discharge; and feeling of facial ‘fullness’ worsening on bending over. In a small number of cases, chronic maxillary sinusitis can also be brought on by the spreading of bacteria from a dental infection. Chronic hyperplastic eosinophilic sinusitis is a noninfective form of chronic sinusitis.

Ictal headaches are headaches associated with seizure activity.

A “subject” as used herein may be a mammal, such as a human or other mammal. Preferably “subject” means a human subject. Said human is preferably an adult human (i.e. a human that is at least 18 years old). The subject may have been diagnosed with a headache disorder, e.g. diagnosed with migraine. The term “subject” and “patient” are used synonymously herein.

A subject for treatment in accordance with the invention may be a subject that is unsuitable for treatment with a non-BoNT/A clostridial neurotoxin. For example, said subject may be unsuitable for treatment with BoNT/B, BoNT/Cl, BoNT/D, BoNT/E, BoNT/F, BoNT/G, BoNT/X, and/or tetanus neurotoxin (TeNT). Said subject may be a subject that is resistant to treatment with a non-BoNT/A clostridial neurotoxin. Resistance may arise due to development of an immune response to a clostridial neurotoxin, including production of anti-clostridial neurotoxin antibodies, by a subject.

The migraine onset of the subject may have occurred when the subject was ≤50 years of age.

The subject may be a male subject. The subject may be a female subject.

A subject may be experiencing one or more symptoms of a headache disorder at the time of administration.

Alternatively, a subject may be one that has been diagnosed with a headache disorder but is not experiencing one or more symptoms of the headache disorder at the time of administration. In such instances, treatment may be prophylactic.

The term “treat” or “treating” as used herein encompasses prophylactic treatment (e.g. to prevent onset of a headache disorder or a symptom thereof) as well as corrective treatment (e.g. treatment of a subject already suffering from a headache disorder or a symptom thereof). Prophylactic treatment may also be referred to as preventive treatment. Preferably, treatment in accordance with the present invention is prophylactic (e.g. preventive treatment).

The term “disorder” as used herein also encompasses a “disease”. For example, the disorder may be a disease.

Treatment of a headache disorder herein encompasses treating (e.g. including preventing) at least one symptom of a headache disorder. Thus, a headache disorder will preferably be considered treated when at least one symptom thereof is treated (e.g. including prevented). A symptom may be considered treated if the severity of said symptom is reduced. Said reduction may be a reduction of at least 5%, 10%, 15%, 20%, 25%, 30%, 35%, 40%, 45%, 50%, 55%, 60%, 65%, 70%, 75%, 80%, 85%, 90%, or 95%, preferably a 100% reduction. The severity of a symptom after treatment may be assessed by comparison to the severity of the same symptom before treatment. The severity of a symptom may be reported by the subject, e.g. by way of a headache diary.

In some instances, treatment of a headache disorder may be considered effective when a subject experiences fewer headache days (e.g. migraine days) per month after treatment in accordance with the invention. This may be determined by comparison with the number of headache days (e.g. migraine days) experienced per month before treatment, such as the average number of headache days (e.g. migraine days) experienced per month before treatment. The average may be a yearly average. For example, treatment of migraine may be considered effective when a subject experiences fewer monthly migraine days and/or monthly headache days after treatment when compared to the monthly migraine days and/or monthly headache days before treatment.

Preferably, the number of headache and/or migraine days per month is assessed after at least 2 treatment sessions in accordance with the invention, e.g. wherein the treatment sessions are 12 weeks apart and wherein the number of headache and/or migraine days per month is assessed 24 weeks after the first treatment session (e.g. based on the number of headache and/or migraine days in the month preceding 24 weeks after the first treatment session, i.e. the number of headache and/or migraine days between week 20 and 24). In some instances, when the number of headache and/or migraine days per month is reduced when compared to the number of headache and/or migraine days per month before the first treatment session (e.g. for a 4 week period before the first treatment session), the headache disorder (e.g. migraine) may be considered effectively treated. For example, the assessment may take into account the number of headache and/or migraine days per month after the second treatment session (e.g. for up to 12 weeks), which may be compared to the number of headache and/or migraine days per month before the first treatment session. Alternatively, the assessment may take into account the number of headache and/or migraine days per month for the entire period following the first treatment session (e.g. for a 24 week period following the first treatment session, where the second treatment session is 12 weeks after the first treatment session), which may be compared to the number of headache and/or migraine days per month before the first treatment session (e.g. for a 4 week period before the first treatment session).

More preferably, when the headache disorder is a migraine, the number of migraine days per month may be assessed after 2 treatment sessions in accordance with the invention, wherein the treatment sessions are 12 weeks apart and wherein the number of migraine days per month is assessed between week 20 and 24 after the first treatment session. In some instances, when the number of migraine days between week 20 and 24 is reduced when compared to the number of migraine days in the 4 weeks immediately preceding the first treatment session (day 1), the migraine may be considered effectively treated.

Any comparison of monthly migraine days and/or monthly headache days may be based on a percentage change or absolute change. For example, the number of headache days and/or migraine days per month experienced by a subject may be reduced by at least 10%, 20%, 30%, 40%, or 50% after treatment in accordance with the invention. For example, the number of headache days and/or migraine days per month experienced by a subject may be reduced by at least 1, 1.5, 2, 3, 4, 5, 6, 7, 8, 9, 10, 11, 12, 13, 14, 15, 16, 17, 18, 19, 20, 21, 22, 23, 24, 25, 26, 27, or 28 days. Said reduction may be evident over a period of at least 4, 8, or 10 weeks. Said reduction may be evident over a period of up to 4 weeks, 8 weeks, 12 weeks, 16 weeks, or 20 weeks, preferably up to 12 weeks. Said reduction may be evident over a period of 4-20 weeks, 6-18 weeks, or 10-14 weeks, preferably 11-13 weeks, more preferably 12 weeks. Preferably, the reduction is a statistically-significant reduction.

A headache day may be a day on which any symptom of a headache disorder is experienced by the subject. In particular, a migraine day may be a day on which any symptom of a migraine is experienced by the subject.

Preferably, the symptom of a headache disorder is headache pain. Thus, in accordance with the invention, a BoNT/A preferably treats (e.g. including prevents) at least headache pain. BoNT/A may treat (e.g. including prevent) one or more symptoms of a headache disorder. Said one or more symptoms may be associated with secretion from a neuron, e.g. release of a mediator, described herein. For example, the mediator CGRP may be involved in a number of symptoms associated with migraine, such as photophobia. Thus, treatment of migraine in accordance with the present invention may treat (e.g. including prevent) one or more symptoms of migraine, such as photophobia and/or pain.

The headache pain mentioned is preferably migraine pain. Said migraine pain may be episodic migraine pain or chronic migraine pain, e.g. pain caused by or otherwise associated with episodic migraine or pain caused by or otherwise associated with chronic migraine.

A BoNT/A may target afferent neurons, thereby resulting in an attenuation of a headache (e.g. migraine) trigger.

A BoNT/A may be administered to a subject in a therapeutically effective amount or a prophylactically effective amount.

A “therapeutically effective amount” is any amount of the BoNT/A, which when administered alone or in combination with another agent (preferably alone) to a subject for treating said headache disorder (e.g. a symptom thereof) is sufficient to effect such treatment of said headache disorder (e.g. a symptom thereof).

A “prophylactically effective amount” is any amount of the BoNT/A that, when administered alone or in combination with another agent (preferably alone) to a subject, inhibits or delays the onset or reoccurrence of a headache disorder (e.g. a symptom thereof). In some embodiments, the prophylactically effective amount prevents the onset or reoccurrence of the headache disorder (e.g. a symptom thereof) entirely. “Inhibiting” the onset means either lessening the likelihood of onset of a headache disorder (e.g. a symptom thereof), preventing the magnitude of the peak effect of the headache disorder (e.g. a symptom thereof), and/or preventing the onset entirely.

The unit doses and/or total doses (preferably total doses) disclosed herein may be suitable therapeutically and/or prophylactically effective amounts.

In some examples, BoNT/A may treat a headache disorder without treating an underlying disorder causing said headache disorder.

A BoNT/A may be able to exert an effect at a site distal to a site of administration (e.g. injection). For example, following administration of the BoNT/A, SNAP25 cleavage may occur at a site distal to the site of administration (e.g. injection). Preferably, such an effect occurs via neuronal transport of the BoNT/A from its site of administration to the distal site. The BoNT/A preferably exerts an effect at a site distal to the site of administration (e.g. injection). This effect may be additional to a peripheral effect. Accordingly, preferably, the BoNT/A may be transported via neuronal transport. Neuronal transport may be retrograde transport or anterograde transport, preferably retrograde transport. The transport may be axonal transport.

“Retrograde transport” may be a form of axonal transport (aka. axoplasmic transport or axoplasmic flow); a cellular process normally responsible for movement of mitochondria, lipids, synaptic vesicles, proteins, and other organelles to and from a neuron's cell body, through the cytoplasm of its axon called the axoplasm. Axons are on the order of meters long, such that neurons cannot rely on diffusion to carry products of the nucleus and organelles to the end of their axons, hence the use of axonal transport. Axonal transport may also be responsible for moving molecules destined for degradation from the axon back to the cell body, where they are broken down by lysosomes.

“Retrograde transport” may refer to movement toward the cell body of a neuron and “anterograde transport” may refer to movement toward the synapse of a neuron.

Neuronal (e.g. retrograde) transport to a neuron of the central nervous system may refer to transport (e.g. axonal transport) of the BoNT/A toward a neuron cell body that is positioned in the proximity of the central nervous system.

Neuronal (e.g. retrograde) transport is now described in more detail. A BoNT/A may bind to a first neuron (such as a primary sensory afferent) at a site of administration. The BoNT/A may be internalised by the first neuron, transported within the first neuron, and then released from the first neuron. Preferably, the BoNT/A binds to a first neuron at a site of intramuscular administration (e.g. intramuscular injection). Such a neuron may be a peripheral neuron. Once released, the BoNT/A may bind to a second neuron, be internalised, and cleave SNAP25 within said second neuron. Alternatively, the BoNT/A may bind to the second neuron, be internalised by the second neuron, transported within the second neuron, and then released from the second neuron. This process may be repeated until the BoNT/A binds to a neuron (e.g. a third neuron), is internalised, and cleaves SNAP25 within said neuron. A second neuron may be a secondary sensory afferent. Preferably, a second neuron is a neuron of the central nervous system, such as a neuron present in the brain, brainstem, or spinal cord. The second neuron may be a neuron present in the trigeminal ganglia (e.g. and SNAP25 cleavage may occur in an axon thereof).

In some examples, when administered intramuscularly, the BoNT/A may be neuronally (e.g. retrogradely) transported via a motor neuron, released from the motor neuron, and enter a second neuron, preferably a neuron of the central nervous system. Said neuron may be a sensory neuron.

In some examples, when administered intramuscularly, the BoNT/A may diffuse to and bind to a sensory neuron present in the periosteum or skin (e.g. terminating in the periosteum or skin).

Without wishing to be bound by theory, it is believed that, by the neuronal (e.g. retrograde) transport mechanism referred to above, the BoNT/A may inhibit secretion from one or more neurons of the central nervous system. Accordingly, the BoNT/A may travel by neuronal (e.g. retrograde) transport to a neuron of the central nervous system and cleave SNAP25 of said neuron.

In a preferred embodiment, by inhibiting secretion (e.g. inhibiting release of a mediator from one or more neuron(s) of the central nervous system), the BoNT/A may treat a headache disorder, preferably migraine. This may be particularly relevant in the treatment of headache pain (preferably migraine pain). The BoNT/A may travel by neuronal (e.g. retrograde) transport to the neuron of the central nervous system and cleave SNAP25 of said neuron. Accordingly, the BoNT/A may treat a headache disorder by inhibiting secretion from a neuron of the central nervous system, preferably by inhibiting secretion of a mediator from a neuron of the central nervous system.

A neuron of the central nervous system may be a neuron of the brainstem, spinal cord, and/or brain.

For example, a neuron of the central nervous system may be a neuron of the: trigeminal nuclei (e.g. the spinal trigeminal nucleus, such as the spinal trigeminal sensory nucleus), spinal cord (preferably a neuron of the dorsal horn of the spinal cord), hypothalamus, thalamus, periaqueductal grey, superior colliculi, inferior colliculi, amygdala, trigeminocervical complex, cortex, and/or the cerebellum. A neuron of the trigeminal nuclei may be a neuron of the trigeminal nucleus caudalis (e.g. pars caudalis).

Preferably, the BoNT/A cleaves SNAP25 in a neuron of the brainstem, more preferably a neuron of the trigeminal nuclei (even more preferably the spinal trigeminal (sensory) nucleus). The BoNT/A may inhibit secretion (e.g. of a mediator) from said neuron. Cleavage of said SNAP25 may occur via neuronal (e.g. retrograde) transport of the BoNT/A from the site of administration.

Such a neuron may be targeted by administering the BoNT/A to or at muscles, the periosteum and/or skin innervated by sensory trigeminal neurons (e.g. muscles, the periosteum, and/or skin located in the face and/or scalp of a subject). Alternatively, said cleavage may be at a neuronal terminal present in the spinal trigeminal sensory nuclei. Most preferably, said SNAP25 cleavage and inhibition of secretion results in the treatment of a headache disorder (e.g. by treating headache pain).

In another preferred embodiment, the BoNT/A cleaves SNAP25 in a neuron of the spinal cord, such as the cervical spinal cord. More preferably, said neuron is a neuron present in the dorsal horn (e.g. associated with sensory neurons) of the spinal cord. The BoNT/A may inhibit secretion (e.g. of a mediator) from said neuron. Cleavage of said SNAP25 may occur via neuronal (e.g. retrograde) transport of the BoNT/A from the site of administration. Such a neuron may be targeted by administering the BoNT/A to or at muscles, the periosteum and/or skin innervated by sensory spinal neurons (e.g. muscles, the periosteum, and/or skin located at the back of head and/or neck of a subject). Alternatively, the neuron may be a sensory afferent and the BoNT/A may cleave SNAP25 thereof (e.g. following transport/diffusion through the cytoplasm of the neuron). Said cleavage may be at a neuronal terminal present in the spinal cord. Most preferably, said SNAP25 cleavage and inhibition of secretion results in the treatment of a headache disorder (e.g. by treating headache pain).

The neuronal (e.g. retrograde) transport of clostridial neurotoxins has been described (see Bomba-Warczak et al (2016), Cell Rep., 16(7), 1974-1987) and, without wishing to be bound by theory, is believed to occur by binding of a BoNT/A to a non-canonical receptor (e.g. in the present case via binding to a receptor other than SV2), incorporation into a non-acidified organelle, neuronal (e.g. retrograde) transport (e.g. away from the periphery of the body towards the central nervous system), and release from the neuron into the extracellular space. In such instances, it has been described that the BoNT/A may remain intact (i.e. the di-chain comprising an L-chain and H-chain joined together by a di-sulphide bond remains intact), allowing for binding via a canonical intoxication route to a second neuron (e.g. via SV2 in the context of a BoNT/A).

A BoNT/A administered to a subject may bind to a sensory neuron and inhibit release of a mediator from said neuron. For example, a BoNT/A administered to a subject may bind to a sensory afferent and inhibit release of a mediator from said neuron. Thus, BoNT/A may exert an effect at a site proximal to the site of administration.

A portion of the BoNT/A administered to a subject may bind to a sensory afferent and inhibit release of a mediator from said neuron, and a portion of the BoNT/A may exert an effect at a site distal to the site of administration. The portion that exerts its effect at a site distal to the site of administration may inhibit secretion from a neuron of the central nervous system, preferably inhibit secretion of a mediator (e.g. a neurotransmitter) from a neuron of the central nervous system. The BoNT/A may travel by neuronal (e.g. retrograde) transport to the neuron of the central nervous system and cleave SNAP25 of said neuron.

Neuronal (e.g. retrograde) transport of the BoNT/A may comprise transynaptic movement (e.g. transcytosis) of the BoNT/A from one neuron to another.

Inhibition of secretion from a neuron may be partial or complete inhibition, preferably complete inhibition. For example, the BoNT/A may inhibit at least 40%, 50%, 60%, 70%, 80%, 90%, 95% or 99% of secretion from the neuron. Preferably, the BoNT/A inhibits 100% of secretion from the neuron. The secretion in this context is preferably SNAP25-associated secretion.

Preferably, a BoNT/A may treat a headache disorder by inhibiting release of a mediator from a sensory afferent and by inhibiting secretion (e.g. of a mediator) from a neuron of the central nervous system.

Preferably, when treating a headache disorder in accordance with the invention, the treatment does not induce muscle paralysis. For example, in some embodiments, the dose (e.g. unit dose or multiple thereof) of the BoNT/A may be lower than the dose (e.g. unit dose or multiple thereof) of the BoNT/A required to induce muscle paralysis. The dose (e.g. unit dose or multiple thereof) of the BoNT/A administered at a particular site (e.g. injection site) may be lower than the dose (e.g. unit dose or multiple thereof) of the BoNT/A required to induce muscle paralysis (e.g. at that site and/or muscle).

The BoNT/A is preferably administered iteratively (e.g. up to 5, 10, 15 or 20 times) as part of a treatment regimen (preferably on different days, e.g. with at least 1 day between successive treatments). A treatment regimen may encompass multiple treatment sessions (individual treatment sessions) referred to herein. Iterative administration means administration at least two times, e.g. at least 5, 10, 15 or 20 times. Thus, a BoNT/A may be administered two or more times to treat a headache disorder of a subject. This is particularly pertinent for the treatment of a chronic headache disorder, such as chronic migraine, where ongoing treatment is typically necessary. A BoNT/A may be administered weekly, twice monthly, monthly, every two months, every six months or annually, preferably at least twice annually or annually. A BoNT/A may be administered two or more times in a period of 10 years, 5 years, 2 years or 1 year. Preferably, a BoNT/A is administered two or more times in a period of 1 year. Treatment may continue for at least 6 months, 1 year, 2 years, 3 years, 5 years, 10 years, 15 years, 20 years, 25 years or 30 years.

Following a first administration of (e.g. first treatment session with) a BoNT/A in accordance with the invention, a subject may be subjected to a second administration of (e.g. second treatment session with) the BoNT/A. The time interval between the first and second administration may be at least 4, 8, or 10 weeks. For example, the time interval between the first and second administration may be 4-20 weeks, 6-18 weeks, or 10-14 weeks, preferably 11-13 weeks (e.g. 12 weeks).

A time interval between any administration may be at least 4, 8, or 10 weeks. For example, the time interval between administrations may be 4-20 weeks, 6-18 weeks, or 10-14 weeks, preferably 11-13 weeks (e.g. 12 weeks).

A time interval of 11-13 weeks (e.g. 12 weeks) may correlate well with the pharmacological effects of abobotulinumtoxinA (Dysport®) following intramuscular injection. Advantageously, this may balance the maintenance of therapeutic coverage with an adequate administration interval for the safety profile. This may also minimise a waning of effect prior to the next administration which may be associated with longer time intervals.

It is preferred that a BoNT/A is not administered together with a further therapeutic or diagnostic agent (e.g. a nucleic acid, protein, peptide or small molecule therapeutic or diagnostic agent) additional to the BoNT/A L-chain and H-chain. For example, in one embodiment a BoNT/A is not administered with a further analgesic. In one embodiment a BoNT/A is not administered together with a covalently associated therapeutic agent. In one embodiment a BoNT/A is not administered together with a non-covalently associated therapeutic agent.

The term “pain” as used here, means any unpleasant sensory and emotional experience associated with, or resembling that associated with, actual or potential tissue damage. Any associated physical disorder may or may not be apparent to a clinician.

Headache pain may be associated with release of a mediator (e.g. a neurotransmitter) from a neuron. The neuron is preferably a neuron in which SNAP25 is cleaved by a BoNT/A. For example, a mediator may be any mediator associated with pain transmission. Thus, a mediator is preferably a pain mediator. A mediator may be a neuropeptide, such as substance P, CGRP, or vasoactive intestinal peptide (VIP). A mediator may be an inflammatory mediator or a non-inflammatory mediator. A mediator may be one or more of: CGRP, a neurokinin (e.g. a tachykinin, substance P, neurokinin A, neurokinin B, a hemokinin and/or an endokinin), adrenocorticotropic hormone (ACTH), glucocorticoids, vasopressin, oxytocin, a catecholamine, an opioid (e.g. an opioid peptide and/or a brain opioid), an angiotensin (e.g. angiotensin II), an endorphin, an encephalin, vasoactive intestinal peptide (VIP), an eicosanoid (e.g. a prostaglandin such as prostaglandin E2 (PGE2), and/or a leukotriene), a tissue kininogen (e.g. bradykinin), histamine, serotonin, potassium, prostacyclin (PGI2), leukotriene B4 (LTB4), nerve growth factor (NGF), protons, ATP, adenosine, 5-hydroxytryptamine (5-HT), histamine, glutamate, norepinephrine (NE), nitric oxide (NO), γ-aminobutyric acid (GABA), glycine, acetylcholine, a cannabinoid, tissue necrosis factor alpha (TNF-α), a cytokine (e.g. interleukin (IL)-6, IL-1, and/or IL-8), a platelet activating factor (PAF), a neurotrophic growth factor (NGF), aspartate, pituitary adenylate cyclase-activating peptide (PACAP), a proteolytic enzyme, amylin, neuropeptide Y (NPY), corticotropin releasing hormone (CRH), leptin, adiponectin, an orexin, and/or melanin-concentrating hormone (MCH). Preferably, the mediator may be one or more of: CGRP, VIP, PACAP, and a proinflammatory cytokine (e.g. IL-6, IL-8, and/or TNF-α). A mediator may be CGRP, substance P and/or an alternative neurokinin. The CGRP may be α-CGRP or β-CGRP, preferably α-CGRP.

A BoNT/A may inhibit the release of a plurality of mediators from a neuron.

The inhibition of release of a mediator from a neuron may be partial or complete inhibition, preferably complete inhibition. For example, the BoNT/A may inhibit at least 40%, 50%, 60%, 70%, 80%, 90%, 95% or 99% of the mediator being released from a neuron. Preferably, the BoNT/A inhibits 100% of the mediator being released from the neuron. The inhibition is preferably inhibition of SNAP25-associated release.

Headache pain may be chronic or acute. An “acute pain” is a pain of short duration having a sudden onset. “Chronic pain” is a pain other than an acute pain. Thus, the headache pain may be chronic or acute headache pain.

A BoNT/A preferably has analgesic properties. In other words, a BoNT/A is preferably an analgesic BoNT/A.

Preferably, a BoNT/A neither promotes neuronal growth nor neuronal repair to treat a headache disorder. In other words, preferably, the BoNT/A does not treat a headache disorder by any of the following means: by promoting neuronal growth, by promoting neuronal repair, or by promoting neuronal growth and repair.

Preferably, a BoNT/A neither promotes neuronal growth nor neuronal repair to treat a headache disorder described herein. In other words, preferably, the BoNT/A does not treat a headache disorder described herein by any of the following means: by promoting neuronal growth, by promoting neuronal repair, or by promoting neuronal growth and repair.

The term “promotes neuronal growth and/or neuronal repair” encompasses an increase in the rate of neuronal growth and/or neuronal repair. The term “neuronal growth and/or neuronal repair” encompasses the rebuilding of damaged neuronal circuits, thereby restoring activity and/or neuronal communication in a network or population of neurons. Thus, the term “neuronal repair” as used herein encompasses repair of a specific neuron as well as repair of a neuronal circuit. The term also encompasses neuronal plasticity. The term “neuronal plasticity” as used herein encompasses axonal sprouting, dendritic sprouting, neurogenesis (e.g. the production of new neurons), maturation, differentiation, and/or synaptic plasticity (e.g. including changes to synaptic strength, activity, anatomy, and/or connectivity). The term “promotes neuronal growth and/or neuronal repair” also encompasses promoting the establishment of functional synapses (e.g. at or near to a site of injury). The term “neuronal growth” as used herein encompasses growth of any part of a neuron, including growth of axons and/or dendrites. Said term encompasses an increase in neurite length, neurite number (e.g. number of neurites per cell), and/or an increase in the length and/or numbers of projections from a cell body or cell membrane of a neuron, e.g. axonal growth of a neuron and/or axonal sprouting, e.g. a neuron in a subject. Said axonal growth may promote connections and/or chemical communication between neurons.

Preferably, a BoNT/A does not promote a neuroimmune response to treat a headache disorder described herein. A neuroimmune response in this context encompasses a microglial response.

Thus, in one embodiment a BoNT/A does not promote a microglial response to treat a disorder described herein.

In a preferred embodiment, the headache disorder described herein is not a disorder associated with, or caused by, a brain disorder. The term “brain disorder” used in this context is interchangeable with “brain disease”. A “brain disorder” as used in this context encompasses a disorder that originates from within or outside the brain, and includes disorders associated with bodily insults that cause brain tissue damage. Examples of brain disorders encompassed in this context include any one (or more) of traumatic brain injury, cancer (e.g. a brain tumour), infectious disease (e.g. encephalitis, meningitis, a brain abscess, and encephalitis), stroke, a neurodegenerative disorder (e.g. Alzheimer's disease, Parkinson's disease, Parkinson's disease related disorders, motor neuron disease (e.g. amyotrophic lateral sclerosis), prion disease, Huntington's disease, spinocerebellar ataxia, ataxia, Hallervorden-Spatz disease, and frontotemporal lobar degeneration), brain aneurysm, multiple sclerosis, anoxic injury, toxic injury and metabolic injury. A brain disorder may be caused by traumatic brain injury, cancer, infectious disease (e.g. encephalitis, meningitis, a brain abscess, and encephalitis), stroke, a neurodegenerative disorder (e.g. Alzheimer's disease, Parkinson's disease, Parkinson's disease related disorders, motor neuron disease (e.g. amyotrophic lateral sclerosis), prion disease, Huntington's disease, spinocerebellar ataxia, ataxia, Hallervorden-Spatz disease, and frontotemporal lobar degeneration), brain aneurysm, multiple sclerosis, anoxic injury, toxic injury and/or metabolic injury.

Embodiments related to the various therapeutic uses of the invention are intended to be applied equally to methods of the invention and vice versa.

### Sequence Homology

Any of a variety of sequence alignment methods can be used to determine percent identity, including, without limitation, global methods, local methods and hybrid methods, such as, e.g., segment approach methods. Protocols to determine percent identity are routine procedures within the scope of one skilled in the art. Global methods align sequences from the beginning to the end of the molecule and determine the best alignment by adding up scores of individual residue pairs and by imposing gap penalties. Non-limiting methods include, e.g., CLUSTAL W, see, e.g., Julie D. Thompson et al., CLUSTAL W: Improving the Sensitivity of Progressive Multiple Sequence Alignment Through Sequence Weighting, Position-Specific Gap Penalties and Weight Matrix Choice, 22(22) Nucleic Acids Research 4673-4680 (1994); and iterative refinement, see, e.g., Osamu Gotoh, Significant Improvement in Accuracy of Multiple Protein. Sequence Alignments by Iterative Refinement as Assessed by Reference to Structural Alignments, 264(4) J. MoI. Biol. 823-838 (1996). Local methods align sequences by identifying one or more conserved motifs shared by all of the input sequences. Non-limiting methods include, e.g., Match-box, see, e.g., Eric Depiereux and Ernest Feytmans, Match-Box: A Fundamentally New Algorithm for the Simultaneous Alignment of Several Protein Sequences, 8(5) CABIOS 501-509 (1992); Gibbs sampling, see, e.g., C. E. Lawrence et al., Detecting Subtle Sequence Signals: A Gibbs Sampling Strategy for Multiple Alignment, 262(5131) Science 208-214 (1993); Align-M, see, e.g., Ivo Van WaIIe et al., Align-M—A New Algorithm for Multiple Alignment of Highly Divergent Sequences, 20(9) Bioinformatics: 1428-1435 (2004).

Thus, percent sequence identity is determined by conventional methods. See, for example, Altschul et al., Bull. Math. Bio. 48: 603-16, 1986 and Henikoff and Henikoff, Proc. Natl. Acad. Sci. USA 89:10915-19, 1992. Briefly, two amino acid sequences are aligned to optimize the alignment scores using a gap opening penalty of 10, a gap extension penalty of 1, and the “blosum 62” scoring matrix of Henikoff and Henikoff (ibid.) as shown below (amino acids are indicated by the standard one-letter codes); preferably this method is used to align a sequence with a subject sequence herein (e.g. SEQ ID NO: 1) to define amino acid position numbering as described herein.

The “percent sequence identity” between two or more nucleic acid or amino acid sequences is a function of the number of identical positions shared by the sequences. Thus, % identity may be calculated as the number of identical nucleotides/amino acids divided by the total number of nucleotides/amino acids, multiplied by 100. Calculations of % sequence identity may also take into account the number of gaps, and the length of each gap that needs to be introduced to optimize alignment of two or more sequences. Sequence comparisons and the determination of percent identity between two or more sequences can be carried out using specific mathematical algorithms, such as BLAST, which will be familiar to a skilled person.

ALIGNMENT SCORES FOR DETERMINING SEQUENCE IDENTITY

| | A | R | N | D | C | Q | E | G | H | I | L | K | M | F | P | S | T | W | Y | V |
|---|---|---|---|---|---|---|---|---|---|---|---|---|---|---|---|---|---|---|---|---|
| A | 4 | | | | | | | | | | | | | | | | | | | |
| R | −1 | 5 | | | | | | | | | | | | | | | | | | |
| N | −2 | 0 | 6 | | | | | | | | | | | | | | | | | |
| D | −2 | −2 | 1 | 6 | | | | | | | | | | | | | | | | |
| C | 0 | −3 | −3 | −3 | 9 | | | | | | | | | | | | | | | |
| Q | −1 | 1 | 0 | 0 | −3 | 5 | | | | | | | | | | | | | | |
| E | −1 | 0 | 0 | 2 | −4 | 2 | 5 | | | | | | | | | | | | | |
| G | 0 | −2 | 0 | −1 | −3 | −2 | −2 | 6 | | | | | | | | | | | | |
| H | −2 | 0 | 1 | −1 | −3 | 0 | 0 | −2 | 8 | | | | | | | | | | | |
| I | −1 | −3 | −3 | −3 | −1 | −3 | −3 | −4 | −3 | 4 | | | | | | | | | | |
| L | −1 | −2 | −3 | −4 | −1 | −2 | −3 | −4 | −3 | 2 | 4 | | | | | | | | | |
| K | −1 | 2 | 0 | −1 | −3 | 1 | 1 | −2 | −1 | −3 | −2 | 5 | | | | | | | | |
| M | −1 | −1 | −2 | −3 | −1 | 0 | −2 | −3 | −2 | 1 | 2 | −1 | 5 | | | | | | | |
| F | −2 | −3 | −3 | −3 | −2 | −3 | −3 | −3 | −1 | 0 | 0 | −3 | 0 | 6 | | | | | | |
| P | −1 | −2 | −2 | −1 | −3 | −1 | −1 | −2 | −2 | −3 | −3 | −1 | −2 | −4 | 7 | | | | | |
| S | 1 | −1 | 1 | 0 | −1 | 0 | 0 | 0 | −1 | −2 | −2 | 0 | −1 | −2 | −1 | 4 | | | | |
| T | 0 | −1 | 0 | −1 | −1 | −1 | −1 | −2 | −2 | −1 | −1 | −1 | −1 | −2 | −1 | 1 | 5 | | | |
| W | −3 | −3 | −4 | −4 | −2 | −2 | −3 | −2 | −2 | −3 | −2 | −3 | −1 | 1 | −4 | −3 | −2 | 11 | | |
| Y | −2 | −2 | −2 | −3 | −2 | −1 | −2 | −3 | 2 | −1 | −1 | −2 | −1 | 3 | −3 | −2 | −2 | 2 | 7 | |
| V | 0 | −3 | −3 | −3 | −1 | −2 | −2 | −3 | −3 | 3 | 1 | −2 | 1 | −1 | −2 | −2 | 0 | −3 | −1 | 4 |

The percent identity is then calculated as:

$$\frac{Total number of identical matches}{[ length of the longer sequence plus the \\ number of gaps introduced into the longer \\ sequence in order to align the two sequences ]} \times 100$$

Substantially homologous polypeptides are characterized as having one or more amino acid substitutions, deletions or additions. These changes are preferably of a minor nature, that is conservative amino acid substitutions (see below) and other substitutions that do not significantly affect the folding or activity of the polypeptide; small deletions, typically of one to about 30 amino acids; and small amino- or carboxyl-terminal extensions, such as an amino-terminal methionine residue, a small linker peptide of up to about 20-25 residues, or an affinity tag.

### Conservative Amino Acid Substitutions

Basic: arginine
   lysine
   histidine
Acidic: glutamic acid
   aspartic acid
Polar: glutamine
   asparagine
Hydrophobic: leucine
   isoleucine
   valine
Aromatic: phenylalanine
   tryptophan
   tyrosine
Small: glycine
   alanine
   serine
   threonine
   methionine

In addition to the 20 standard amino acids, non-standard amino acids (such as 4-hydroxyproline, 6-N-methyl lysine, 2-aminoisobutyric acid, isovaline and α-methyl serine) may be substituted for amino acid residues of the polypeptides of the present invention. A limited number of non-conservative amino acids, amino acids that are not encoded by the genetic code, and unnatural amino acids may be substituted for polypeptide amino acid residues. The polypeptides of the present invention can also comprise non-naturally occurring amino acid residues.

Non-naturally occurring amino acids include, without limitation, trans-3-methylproline, 2,4-methano-proline, cis-4-hydroxyproline, trans-4-hydroxy-proline, N-methylglycine, allo-threonine, methyl-threonine, hydroxy-ethylcysteine, hydroxyethylhomo-cysteine, nitro-glutamine, homoglutamine, pipecolic acid, tert-leucine, norvaline, 2-azaphenylalanine, 3-azaphenyl-alanine, 4-azaphenyl-alanine, and 4-fluorophenylalanine. Several methods are known in the art for incorporating non-naturally occurring amino acid residues into proteins. For example, an in vitro system can be employed wherein nonsense mutations are suppressed using chemically aminoacylated suppressor tRNAs. Methods for synthesizing amino acids and aminoacylating tRNA are known in the art. Transcription and translation of plasmids containing nonsense mutations is carried out in a cell free system comprising an E. coli S30 extract and commercially available enzymes and other reagents. Proteins are purified by chromatography. See, for example, Robertson et al., J. Am. Chem. Soc. 113:2722, 1991; Ellman et al., Methods Enzymol. 202:301, 1991; Chung et al., Science 259:806-9, 1993; and Chung et al., Proc. Natl. Acad. Sci. USA 90:10145-9, 1993). In a second method, translation is carried out in Xenopus oocytes by microinjection of mutated mRNA and chemically aminoacylated suppressor tRNAs (Turcatti et al., J. Biol. Chem. 271:19991-8, 1996). Within a third method, E. coli cells are cultured in the absence of a natural amino acid that is to be replaced (e.g., phenylalanine) and in the presence of the desired non-naturally occurring amino acid(s) (e.g., 2-azaphenylalanine, 3-azaphenylalanine, 4-azaphenylalanine, or 4-fluorophenylalanine). The non-naturally occurring amino acid is incorporated into the polypeptide in place of its natural counterpart. See, Koide et al., Biochem. 33:7470-6, 1994. Naturally occurring amino acid residues can be converted to non-naturally occurring species by in vitro chemical modification. Chemical modification can be combined with site-directed mutagenesis to further expand the range of substitutions (Wynn and Richards, Protein Sci. 2:395-403, 1993).

A limited number of non-conservative amino acids, amino acids that are not encoded by the genetic code, non-naturally occurring amino acids, and unnatural amino acids may be substituted for amino acid residues of polypeptides of the present invention.

Essential amino acids in the polypeptides of the present invention can be identified according to procedures known in the art, such as site-directed mutagenesis or alanine-scanning mutagenesis (Cunningham and Wells, Science 244: 1081-5, 1989). Sites of biological interaction can also be determined by physical analysis of structure, as determined by such techniques as nuclear magnetic resonance, crystallography, electron diffraction or photoaffinity labeling, in conjunction with mutation of putative contact site amino acids. See, for example, de Vos et al., Science 255:306-12, 1992; Smith et al., J. Mol. Biol. 224:899-904, 1992; Wlodaver et al., FEBS Lett. 309:59-64, 1992. The identities of essential amino acids can also be inferred from analysis of homologies with related components (e.g. the translocation or protease components) of the polypeptides of the present invention.

Multiple amino acid substitutions can be made and tested using known methods of mutagenesis and screening, such as those disclosed by Reidhaar-Olson and Sauer (Science 241:53-7, 1988) or Bowie and Sauer (Proc. Natl. Acad. Sci. USA 86:2152-6, 1989). Briefly, these authors disclose methods for simultaneously randomizing two or more positions in a polypeptide, selecting for functional polypeptide, and then sequencing the mutagenized polypeptides to determine the spectrum of allowable substitutions at each position. Other methods that can be used include phage display (e.g., Lowman et al., Biochem. 30:10832-7, 1991; Ladner et al., U.S. Pat. No. 5,223,409; Huse, WIPO Publication WO 92/06204) and region-directed mutagenesis (Derbyshire et al., Gene 46:145, 1986; Ner et al., DNA 7:127, 1988).

Unless defined otherwise, all technical and scientific terms used herein have the same meaning as commonly understood by one of ordinary skill in the art to which this disclosure belongs. Singleton, et al., DICTIONARY OF MICROBIOLOGY AND MOLECULAR BIOLOGY, 20 ED., John Wiley and Sons, New York (1994), and Hale & Marham, THE HARPER COLLINS DICTIONARY OF BIOLOGY, Harper Perennial, NY (1991) provide the skilled person with a general dictionary of many of the terms used in this disclosure.

This disclosure is not limited by the exemplary methods and materials disclosed herein, and any methods and materials similar or equivalent to those described herein can be used in the practice or testing of embodiments of this disclosure. Numeric ranges are inclusive of the numbers defining the range. Unless otherwise indicated, any nucleic acid sequences are written left to right in 5′ to 3′ orientation; amino acid sequences are written left to right in amino to carboxy orientation, respectively.

The headings provided herein are not limitations of the various aspects or embodiments of this disclosure.

Amino acids are referred to herein using the name of the amino acid, the three letter abbreviation or the single letter abbreviation. The term “protein”, as used herein, includes proteins, polypeptides, and peptides. As used herein, the term “amino acid sequence” is synonymous with the term “polypeptide” and/or the term “protein”. In some instances, the term “amino acid sequence” is synonymous with the term “peptide”. In some instances, the term “amino acid sequence” is synonymous with the term “enzyme”. The terms “protein” and “polypeptide” are used interchangeably herein. In the present disclosure and claims, the conventional one-letter and three-letter codes for amino acid residues may be used. The 3-letter code for amino acids as defined in conformity with the IUPACIUB Joint Commission on Biochemical Nomenclature (JCBN). It is also understood that a polypeptide may be coded for by more than one nucleotide sequence due to the degeneracy of the genetic code.

Other definitions of terms may appear throughout the specification. Before the exemplary embodiments are described in more detail, it is to be understood that this disclosure is not limited to particular embodiments described, and as such may vary. It is also to be understood that the terminology used herein is for the purpose of describing particular embodiments only, and is not intended to be limiting, since the scope of the present disclosure will be defined only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within this disclosure. The upper and lower limits of these smaller ranges may independently be included or excluded in the range, and each range where either, neither or both limits are included in the smaller ranges is also encompassed within this disclosure, subject to any specifically excluded limit in the stated range. Where the stated range includes one or both of the limits, ranges excluding either or both of those included limits are also included in this disclosure.

It must be noted that as used herein and in the appended claims, the singular forms “a”, “an”, and “the” include plural referents unless the context clearly dictates otherwise. Thus, for example, reference to “a botulinum neurotoxin” includes a plurality of such candidate agents and reference to “the botulinum neurotoxin” includes reference to one or more botulinum neurotoxins and equivalents thereof known to those skilled in the art, and so forth.

The publications discussed herein are provided solely for their disclosure prior to the filing date of the present application. Nothing herein is to be construed as an admission that such publications constitute prior art to the claims appended hereto.

### BRIEF DESCRIPTION OF THE DRAWINGS

Embodiments of the invention will now be described, by way of example only, with reference to the following Figures and Examples. Many of the Figures submitted herein are better understood in colour. The colour versions of the drawings are part of the application as filed and the right to present colour images of the drawings in later proceedings is hereby reserved.

FIG. 1 shows the proportion (%) of the area of the spinal trigeminal sensory nuclei in the brainstem that stained positive for cleaved SNAP25 (c-SNAP25) in rats administered BoNT/A.

FIG. 2 shows the amount of cleaved SNAP25 (c-SNAP25) in the dorsal horn (sensory) in the cervical spinal cord in rats administered BoNT/A. The amount is represented by way of a scoring system (“c-SNAP25 IHC score”) as explained in Example 1.

FIG. 3 shows the muscles and injection sites treated in accordance with an embodiment of the invention. Injection can only be seen at one temporalis muscle in the central image, however, in this embodiment the equivalent temporalis muscle at the opposite side of the head is also injected in an equivalent manner.

### SEQUENCE LISTING

Where an initial Met amino acid residue is indicated in any of the following SEQ ID NOs, said residue is optional and preferably absent.

### SEQ ID NO: 1—Polypeptide Sequence of BoNT/A

MPFVNKQFNYKDPVNGVDIAYIKIPNAGQMQPVKAFKIHNKIWVIPERDTFTNPEEGDLN PPPEAKQVPVSYYDSTYLSTDNEKDNYLKGVTKLFERIYSTDLGRMLLTSIVRGIPFWGG STIDTELKVIDTNCINVIQPDGSYRSEELNLVIIGPSADIIQFECKSFGHEVLNLTRNGY GSTQYIRFSPDFTFGFEESLEVDTNPLLGAGKFATDPAVTLAHELIHAGHRLYGIAINPN RVFKVNTNAYYEMSGLEVSFEELRTFGGHDAKFIDSLQENEFRLYYYNKFKDIASTLNK A KSIVGTTASLQYMKNVFKEKYLLSEDTSGKFSVDKLKFDKLYKMLTEIYTEDNFVKFFK V LNRKTYLNFDKAVFKINIVPKVNYTIYDGFNLRNTNLAANFNGQNTEINNMNFTKLKNF T GLFEFYKLLCVRGIITSKTKSLDKGYNKALNDLCIKVNNWDLFFSPSEDNFTNDLNKGEE ITSDTNIEAAEENISLDLIQQYYLTFNFDNEPENISIENLSSDIIGQLELMPNIERFPNG KKYELDKYTMFHYLRAQEFEHGKSRIALTNSVNEALLNPSRVYTFFSSDYVKKVNKATE A AMFLGWVEQLVYDFTDETSEVSTTDKIADITIIIPYIGPALNIGNMLYKDDFVGALIFSG AVILLEFIPEIAIPVLGTFALVSYIANKVLTVQTIDNALSKRNEKWDEVYKYIVTNWLAK VNTQIDLIRKKMKEALENQAEATKAIINYQYNQYTEEEKNNINFNIDDLSSKLNESINKA MININKFLNQCSVSYLMNSMIPYGVKRLEDFDASLKDALLKYIYDNRGTLIGQVDRLKD K VNNTLSTDIPFQLSKYVDNQRLLSTFTEYIKNIINTSILNLRYESNHLIDLSRYASKINI GSKVNFDPIDKNQIQLFNLESSKIEVILKNAIVYNSMYENFSTSFWIRIPKYFNSISLNN EYTIINCMENNSGWKVSLNYGEIIWTLQDTQEIKQRVVFKYSQMINISDYINRWIFVTIT NNRLNNSKIYINGRLIDQKPISNLGNIHASNNIMFKLDGCRDTHRYIWIKYFNLFDKELN EKEIKDLYDNQSNSGILKDFWGDYLQYDKPYYMLNLYDPNKYVDVNNVGIRGYMYLK GPR GSVMTTNIYLNSSLYRGTKFIIKKYASGNKDNIVRNNDRVYINVVVKNKEYRLATNASQ A GVEKILSALEIPDVGNLSQVVVMKSKNDQGITNKCKMNLQDNNGNDIGFIGFHQFNNIA K LVASNWYNRQIERSSRTLGCSWEFIPVDDGWGERPL

### SEQ ID NO: 2—Di-Chain Light-Chain 1

PFVNKQFNYKDPVNGVDIAYIKIPNAGQMQPVKAFKIIINKIWVIPERDTFTNPEEGDLN PPPEAKQVPVSYYDSTYLSTDNEKDNYLKGVTKLFERIYSTDLGRMLLTSIVRGIPFWGG STIDTELKVIDTNCINVIQPDGSYRSEELNLVIIGPSADIIQFECKSFGHEVLNLTRNGY GSTQYIRFSPDFTFGFEESLEVDTNPLLGAGKFATDPAVTLAHELIHAGHRLYGIAINPN RVFKVNTNAYYEMSGLEVSFEELRTFGGHDAKFIDSLQENEFRLYYYNKFKDIASTLNK A KSIVGTTASLQYMKNVFKEKYLLSEDTSGKFSVDKLKFDKLYKMLTEIYTEDNFVKFFK V LNRKTYLNFDKAVFKINIVPKVNYTIYDGFNLRNTNLAANFNGQNTEINNMNFTKLKNF T GLFEFYKLLCVRGIITSK

### SEQ ID NO: 3—Di-Chain Light-Chain 2

PFVNKQFNYKDPVNGVDIAYIKIPNAGQMQPVKAFKIHNKIWVIPERDTFTNPEEGDLN PPPEAKQVPVSYYDSTYLSTDNEKDNYLKGVTKLFERIYSTDLGRMLLTSIVRGIPFWGG STIDTELKVIDTNCINVIQPDGSYRSEELNLVIIGPSADIIQFECKSFGGHEVLNLTRNGY GSTQYIRFSPDFTFGFEESLEVDTNPLLGAGKFATDPAVTLAHELIHAGHRLYGIAINPN RVFKVNTNAYYEMSGLEVSFEELRTFGGHDAKFIDSLQENEFRLYYYNKFKDIASTLNK A KSIVGTTASLQYMKNVFKEKYLLSEDTSGKFSVDKLKFDKLYKMLTEIYTEDNFVKFFK V LNRKTYLNFDKAVFKINIVPKVNYTIYDGFNLRNTNLAANFNGQNTEINNMNFTKLKNF T GLFEFYKLLCVRGIITSKTK

### SEQ ID NO: 4—Di-Chain Heavy-Chain

ALNDLCIKVNNWDLFFSPSEDNFTNDLNKGEE ITSDTNIEAAEENISLDLIQQYYLTFNFDNEPENISIENLSSDIIGQLELMPNIERFPNG KKYELDKYTMFHYLRAQEFEHGKSRIALTNSVNEALLNPSRVYTFFSSDYVKKVNKATE A AMFLGWVEQLVYDFTDETSEVSTTDKIADITIIIPYIGPALNIGNMLYKDDFVGALIFSG AVILLEFIPEIAIPVLGTFALVSYIANKVLTVQTIDNALSKRNEKWDEVYKYIVTNWLAK VNTQIDLIRKKMKEALENQAEATKAIINYQYNQYTEEEKNNINFNIDDLSSKLNESINKA MININKFLNQCSVSYLMNSMIPYGVKRLEDFDASLKDALLKYIYDNRGTLIGQVDRLKD K VNNTLSTDIPFQLSKYVDNQRLLSTFTEYIKNIINTSILNLRYESNHLIDLSRYASKINI GSKVNFDPIDKNQIQLFNLESSKIEVILKNAIVYNSMYENFSTSFWIRIPKYFNSISLNN EYTIINCMENNSGWKVSLNYGEIIWTLQDTQEIKQRVVFKYSQMINISDYINRWIFVTIT NNRLNNSKIYINGRLIDQKPISNLGNIHASNNIMFKLDGCRDTHRYIWIKYFNLFDKELN EKEIKDLYDNQSNSGILKDFWGDYLQYDKPYYMLNLYDPNKYVDVNNVGIRGYMYLK GPR GSVMTTNIYLNSSLYRGTKFIIKKYASGNKDNIVRNNDRVYINVVVKNKEYRLATNASQ A GVEKILSALEIPDVGNLSQVVVMKSKNDQGITNKCKMNLQDNNGNDIGFIGFHQFNNIA K LVASNWYNRQIERSSRTLGCSWEFIPVDDGWGERPL

### SEQ ID NO: 5—C-terminal L-Chain Fragment 1

TKSLDKGYNK

### SEQ ID NO: 6—C-terminal L-Chain Fragment 2

SLDKGYNK

### EXAMPLES

### Example 1

### BoNT/a Cleaves SNAP25 in Neurons Relevant for Treating Headache Disorders In Vivo

### Materials & Methods

Naïve female Sprague Dawley rats were used for the study (180-220 g at treatment initiation, Janvier Labs, France). Animals were kept on a reversed 12-h light/dark cycle (lights on from 18:00 to 06:00) and maintained in an enriched environment under a constant temperature (22±2° C.) and humidity (55±5%) with food and water available ad libitum. Animals were acclimatized for at least 7 days prior to experimentation. The study was performed in full compliance with the ARRIVE guidelines, European Communities Council Directive 2010/63/EU and French National Committee decree 87/848.

The BoNT/A used had been isolated from Clostridium botulinum type A and was present together with complexing proteins.

Animals were administered vehicle (saline, 2 rats/group) or BoNT/A (6 rats/group) using intramuscular (IM) injection. IM administrations were performed by dividing the total dose into 4 muscles of the head and neck (right and left temporalis, right and left occipitalis, 10 μL of injection volume each). 10 days after treatment administration, animals were euthanized and the following tissues were harvested: the brainstem comprising the spinal trigeminal nuclei and the cervical spinal cord. Tissues were then fixed in isotonic buffered formalin 10% solution (VWR, France) for 48 h, embedded in paraffin blocks and histologic slides were prepared.

To evaluate the biological effect of BoNT/A in the tissues, an immunohistochemical staining of the cleaved form of SNAP25 (c-SNAP25) was performed. After a heat-induced epitope retrieval step, endogenous peroxidases were blocked for 10 min in a 3% H₂O₂ solution in a TBS buffer. The sections were incubated with a non-commercial primary rabbit polyclonal antibody (EF14007, Ipsen Innovation, France) which is specific for the cleaved form of SNAP25 by BoNT/A only. Sections were then incubated with a biotinylated secondary antibody for 30 min (anti-rabbit IgG, Vector Laboratories, USA), followed by a 30 min incubation with an amplification system (avidin-biotin) coupled to horseradish peroxidase (Vector Laboratories, USA). Finally, sections were incubated for 5 min with a solution of 0.02% diaminobenzidine (DAKO, USA), counterstaining was done using haematoxylin (DAKO, USA), and the slides were visualized under the light microscope.

In the spinal cord, the intensity and density of c-SNAP25 positive nerve endings was graded as follow: 0 (no staining), 1 (minimal), 2 (mild), 3 (moderate), 4 (marked) on the 5 most intensely stained spinal cord sections, a cumulative score (0 to 20) was then calculated for each animal. For brainstem samples, SNAP25 cleavage staining was quantified using a dedicated image analysis method that measures the proportion of nerve fibers stained for c-SNAP25.

### Results

As expected, no specific c-SNAP25 staining was observed in tissues from any vehicle-treated animal.

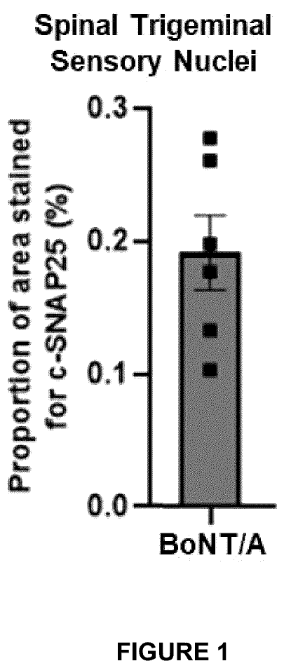

FIG. 1 shows that administration of BoNT/A resulted in SNAP25 cleavage at the spinal trigeminal sensory nuclei of the brainstem.

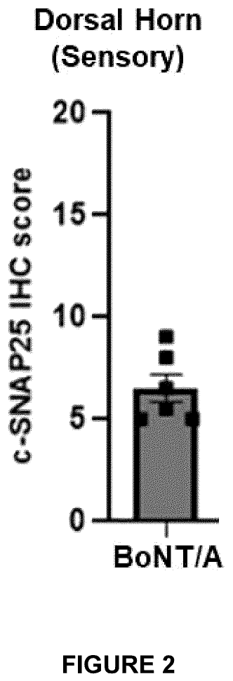

FIG. 2 shows that administration of BoNT/A resulted in SNAP25 cleavage in the sensory dorsal horn of the cervical spinal cord.

In conclusion, BoNT/A has been shown to cleave SNAP25 in key central sensory structures shown to be involved in headache disorders (e.g. migraine). In particular, these data show that BoNT/A is able to travel by neuronal transport from the injection site to neurons of the central nervous system.

The data further confirm the temporalis and occipitalis muscles as advantageous sites of administration.

### Example 2

Treatment of a Patient with Episodic Migraine

Timothy 33 is diagnosed by his GP with episodic migraine. He is treated by way of a unit dose (UD) of 9.09 U Units (U) of abobotulinumtoxinA (Dysport®), where 1 Unit of abobotulinumtoxinA (Dysport®) corresponds to the calculated median lethal intraperitoneal dose (LD50) in mice. The physician adds 3.3 ml of 0.9% Sodium Chloride for Injection (United States Pharmacopoeia—USP) to a 300 U vial containing abobotulinumtoxinA (Dysport®) in a dry powder form, yielding a dose of 9.09 U of toxin per 0.1 ml (rounded to the nearest integer).

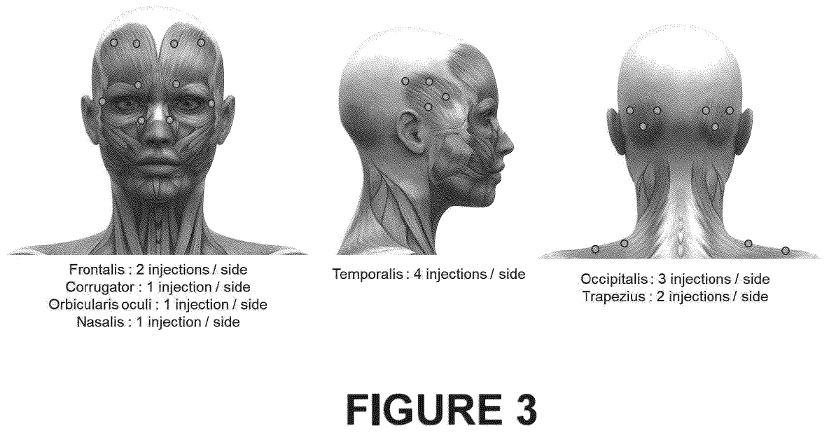

AbobotulinumtoxinA (Dysport®) is administered by intramuscular injection as follows (see FIG. 3 for positions of injection sites):

| | No. of Injection | | |
|---|---|---|---|
| | Sites (0.1 ml | Dose/ | Dose/ |
| | per Site) Per | Side | Session |
| Muscle Injected | Side/Per Session | (U)* | (U)* |
| Frontalis (near hairline) | 2/4 | 18 | 36 |
| Corrugator | 1/2 | 9 | 18 |
| Nasalis | 1/2 | 9 | 18 |
| Orbicularis Oculi | 1/2 | 9 | 18 |
| Temporalis | 4/8 | 36 | 73 |
| Occipitalis | 3/6 | 27 | 55 |
| Trapezius | 2/4 | 18 | 36 |
| Total | 14/28 | 127 | 255 |

*Rounded to the nearest integer, calculation performed with dose per site to two decimal places (9.09 U).

Timothy receives a total of 255 U of abobotulinumtoxinA (Dysport®) during the treatment and experiences fewer migraine days per month following treatment.

### Example 3

Treatment of a Patient with Chronic Migraine

Sally 42 is diagnosed by her GP with chronic migraine. She is treated by way of a unit dose (UD) of 15.15 U Units (U) of abobotulinumtoxinA (Dysport®), where 1 Unit of abobotulinumtoxinA (Dysport®) corresponds to the calculated median lethal intraperitoneal dose (LD50) in mice. The physician adds 3.3 ml of 0.9% Sodium Chloride for Injection (USP) to a 500 U vial containing abobotulinumtoxinA (Dysport®) in a dry powder form, yielding a dose of 15.15 U of toxin per 0.1 ml (rounded to the nearest integer).

AbobotulinumtoxinA (Dysport®) is administered by intramuscular injection in the region of the muscles indicated as follows (see FIG. 3 for positions of injection sites):

| | No. of Injection | | |
|---|---|---|---|
| | Sites (0.1 ml | Dose/ | Dose/ |
| | per Site) Per | Side | Session |
| Muscle | Side/Per Session | (U)* | (U)* |
| Frontalis (near hairline) | 2/4 | 30 | 61 |
| Corrugator | 1/2 | 15 | 30 |
| Nasalis | 1/2 | 15 | 30 |
| Orbicularis Oculi | 1/2 | 15 | 30 |
| Temporalis | 4/8 | 61 | 121 |
| Occipitalis | 3/6 | 45 | 91 |
| Trapezius | 2/4 | 30 | 61 |
| Total | 14/28 | 212 | 424 |

*Rounded to the nearest integer, calculation performed with dose per site to two decimal places (15.15 U).

Sally receives a total of 424 U of abobotulinumtoxinA (Dysport®) during the treatment and experiences fewer migraine days per month following treatment. She is retreated 12 weeks later.

### Example 4

Preventive Treatment of a Patient with Chronic Migraine

Connie is treated by way of a unit dose (UID) of 15.15 U Units (U) of abobotulinumtoxinA (Dysport®), where 1 Unit of abobotulinumtoxinA (Dysport®) corresponds to the calculated median lethal intraperitoneal dose (LD50) in mice. The physician adds 3.3 ml of 0.9% Sodium Chloride for Injection (USP) to a 500 U vial containing abobotulinumtoxinA (Dysport®) in a dry powder form, yielding a dose of 15.15 U of toxin per 0.1 ml (rounded to the nearest integer).

AbobotulinumtoxinA (Dysport®) is administered by intramuscular injection in the region of the muscles indicated as follows (see FIG. 3 for positions of injection sites):

| | No. of Injection | | |
|---|---|---|---|
| | Sites (0.1 ml | Dose/ | Dose/ |
| | per Site) Per | Side | Session |
| Muscle | Side/Per Session | (U)* | (U)* |
| Frontalis (near hairline) | 2/4 | 30 | 61 |
| Corrugator | 1/2 | 15 | 30 |
| Nasalis | 1/2 | 15 | 30 |
| Orbicularis Oculi | 1/2 | 15 | 30 |
| Temporalis | 4/8 | 61 | 121 |
| Occipitalis | 3/6 | 45 | 91 |
| Trapezius | 2/4 | 30 | 61 |
| Total | 14/28 | 212 | 424 |

*Rounded to the nearest integer, calculation performed with dose per site to two decimal places (15.15 U).

Connie receives a total of 424 U of abobotulinumtoxinA (Dysport®) during the treatment and is treated every 12 weeks. During this time, Connie experiences fewer migraine days per month.

All publications mentioned in the above specification are herein incorporated by reference. Various modifications and variations of the described methods and system of the present invention will be apparent to those skilled in the art without departing from the scope and spirit of the present invention. Although the present invention has been described in connection with specific preferred embodiments, it should be understood that the invention as claimed should not be unduly limited to such specific embodiments. Indeed, various modifications of the described modes for carrying out the invention which are obvious to those skilled in biochemistry and biotechnology or related fields are intended to be within the scope of the following claims.

1. A method of treating a headache disorder in a subject, the method comprising administering a botulinum neurotoxin A (BoNT/A) to or at an orbicularis oculi muscle and/or a nasalis muscle of the subject.

2. The method according to claim 1, further comprising administering the BoNT/A to or at:
(a) a frontalis muscle of the subject;
(b) a corrugator muscle of the subject;
(c) a temporalis muscle of the subject;
(d) an occipitalis muscle of the subject; and/or
(e) a trapezius muscle of the subject.

3. A method of treating a headache disorder in a subject by inhibiting secretion from a neuron of the central nervous system, the method comprising administering a botulinum neurotoxin A (BoNT/A) to the subject.

4. (canceled)

5. The method of claim 3, comprising administering the BoNT/A to or at:
(a) a frontalis muscle of the subject;
(b) a corrugator muscle of the subject;
(c) a nasalis muscle of the subject;
(d) an orbicularis oculi muscle of the subject;
(e) a temporalis muscle of the subject;
(f) an occipitalis muscle of the subject; and
(g) a trapezius muscle of the subject.

6. (canceled)

7. The method of claim 1, wherein the BoNT/A travels by neuronal transport to a neuron of the central nervous system and cleaves a synaptosomal-associated protein of 25 kDa (SNAP25) protein of said neuron.

8. The method according to claim 1, wherein the headache disorder is migraine.

9. The method according to claim 5, wherein administration of the BoNT/A comprises administering:
(a) 2 unit doses to or at each frontalis muscle;
(b) 1 unit dose to or at each corrugator muscle;
(c) 1 unit dose to or at each nasalis muscle;
(d) 1 unit dose to or at each orbicularis oculi muscle;
(e) 4 unit doses to or at each temporalis muscle;
(f) 3 unit doses to or at each occipitalis muscle; and
(g) 2 unit doses to or at each trapezius muscle.

10.-13. (canceled)

14. The method according to claim 1, wherein the BoNT/A is administered by way of a unit dose of 4-20 Units (U), wherein 1 Unit corresponds to the calculated median lethal intraperitoneal dose (LD50) in mice.

15. The method according to claim 1, wherein the BoNT/A is BTX-A-HAC.

16. (canceled)

17. The method according to claim 14, wherein the unit dose is 4-14 U, 8-10 U, 10-20 U, or 14-16 U.

18. The method according to claim 14, wherein the unit dose is 9.09 U or 15.15 U.

19.-20. (canceled)

21. The method according to claim 14, wherein the total dose administered is up to 255 U, up to 280 U, up to 392 U, up to 424 U, up to 448 U, or up to 560 U.

22. The method according to claim 14, wherein the total dose administered is 112-392 U, 112-448 U, 112-560 U, 224-280 U, 280-560 U, or 392-448 U.

23.-25. (canceled)

26. The method according to claim 14, wherein the total dose administered is 255 U or 424 U.

27.-28. (canceled)

29. The method according to claim 14, wherein the total dose administered is at least 300 U.

30.-33. (canceled)

34. The method according to claim 1, wherein the administration is via intramuscular injection.

35. The method according to claim 1, wherein the treatment is prophylactic treatment.

36. The method according to claim 1, wherein administration of the BoNT/A comprises:
(a) 4 intramuscular injections of abobotulinumtoxinA to the frontalis muscles via 2 intramuscular injections of abobotulinumtoxinA to a frontalis muscle at a first side of the face and 2 intramuscular injections of abobotulinumtoxinA to a frontalis muscle at a second side of the face;
(b) 2 intramuscular injections of abobotulinumtoxinA to the corrugator muscles via 1 intramuscular injection of abobotulinumtoxinA to a corrugator muscle at a first side of the face and 1 intramuscular injection of abobotulinumtoxinA to a corrugator muscle at a second side of the face;
(c) 2 intramuscular injections of abobotulinumtoxinA to the nasalis muscles via 1 intramuscular injection of abobotulinumtoxinA to a nasalis muscle at a first side of the face and 1 intramuscular injection of abobotulinumtoxinA to a nasalis muscle at a second side of the face;
(d) 2 intramuscular injections of abobotulinumtoxinA to the orbicularis oculi muscles via 1 injection of abobotulinumtoxinA to an orbicularis oculi muscle at a first side of the face and 1 intramuscular injection of abobotulinumtoxinA to an orbicularis oculi muscle at a second side of the face;
(e) 8 intramuscular injections of abobotulinumtoxinA to the temporalis muscles via 4 intramuscular injections of abobotulinumtoxinA to a temporalis muscle at a first side of the head and 4 intramuscular injections of abobotulinumtoxinA to a temporalis muscle at a second side of the head;
(f) 6 intramuscular injections of abobotulinumtoxinA to the occipitalis muscles via 3 intramuscular injections of abobotulinumtoxinA to an occipitalis muscle at a first side of the head and 3 intramuscular injections of abobotulinumtoxinA to an occipitalis muscle at a second side of the head; and
(g) 4 intramuscular injections of abobotulinumtoxinA to the trapezius muscles via 2 intramuscular injections of abobotulinumtoxinA to a trapezius muscle at a first side of the neck and 2 intramuscular injections of abobotulinumtoxinA to a trapezius muscle at a second side of the neck; and
wherein 9.09 U of abobotulinumtoxinA is administered per injection.

37. The method according to claim 1, wherein administration of the BoNT/A comprises:
(a) 4 intramuscular injections of abobotulinumtoxinA to the frontalis muscles via 2 intramuscular injections of abobotulinumtoxinA to a frontalis muscle at a first side of the face and 2 intramuscular injections of abobotulinumtoxinA to a frontalis muscle at a second side of the face;
(b) 2 intramuscular injections of abobotulinumtoxinA to the corrugator muscles via 1 intramuscular injection of abobotulinumtoxinA to a corrugator muscle at a first side of the face and 1 intramuscular injection of abobotulinumtoxinA to a corrugator muscle at a second side of the face;
(c) 2 intramuscular injections of abobotulinumtoxinA to the nasalis muscles via 1 intramuscular injection of abobotulinumtoxinA to a nasalis muscle at a first side of the face and 1 intramuscular injection of abobotulinumtoxinA to a nasalis muscle at a second side of the face;
(d) 2 intramuscular injections of abobotulinumtoxinA to the orbicularis oculi muscles via 1 injection of abobotulinumtoxinA to an orbicularis oculi muscle at a first side of the face and 1 intramuscular injection of abobotulinumtoxinA to an orbicularis oculi muscle at a second side of the face;
(e) 8 intramuscular injections of abobotulinumtoxinA to the temporalis muscles via 4 intramuscular injections of abobotulinumtoxinA to a temporalis muscle at a first side of the head and 4 intramuscular injections of abobotulinumtoxinA to a temporalis muscle at a second side of the head;
(f) 6 intramuscular injections of abobotulinumtoxinA to the occipitalis muscles via 3 intramuscular injections of abobotulinumtoxinA to an occipitalis muscle at a first side of the head and 3 intramuscular injections of abobotulinumtoxinA to an occipitalis muscle at a second side of the head; and
(g) 4 intramuscular injections of abobotulinumtoxinA to the trapezius muscles via 2 intramuscular injections of abobotulinumtoxinA to a trapezius muscle at a first side of the neck and 2 intramuscular injections of abobotulinumtoxinA to a trapezius muscle at a second side of the neck; and
wherein 15.15 U of abobotulinumtoxinA is administered per injection.

38. The method according to claim 1, wherein administration of the BoNT/A comprises administration of the BoNT/A to or at the muscles via injection sites shown in FIG. 3.


SEQUENCE LISTING (6 sequences)

SEQ ID NO: 1 - protein, 1296 aa, Clostridium botulinum
MPFVNKQFNYKDPVNGVDIAYIKIPNAGQMQPVKAFKIHNKIWVIPERDTFTNPEEGDLNPPPEAKQVPVSYYDSTYLSTDNEKDNYLKGVTKLFERIYSTDLGRMLLTSIVRGIPFWGGSTIDTELKVIDTNCINVIQPDGSYRSEELNLVIIGPSADIIQFECKSFGHEVLNLTRNGYGSTQYIRFSPDFTFGFEESLEVDTNPLLGAGKFATDPAVTLAHELIHAGHRLYGIAINPNRVFKVNTNAYYEMSGLEVSFEELRTFGGHDAKFIDSLQENEFRLYYYNKFKDIASTLNKAKSIVGTTASLQYMKNVFKEKYLLSEDTSGKFSVDKLKFDKLYKMLTEIYTEDNFVKFFKVLNRKTYLNFDKAVFKINIVPKVNYTIYDGFNLRNTNLAANFNGQNTEINNMNFTKLKNFTGLFEFYKLLCVRGIITSKTKSLDKGYNKALNDLCIKVNNWDLFFSPSEDNFTNDLNKGEEITSDTNIEAAEENISLDLIQQYYLTFNFDNEPENISIENLSSDIIGQLELMPNIERFPNGKKYELDKYTMFHYLRAQEFEHGKSRIALTNSVNEALLNPSRVYTFFSSDYVKKVNKATEAAMFLGWVEQLVYDFTDETSEVSTTDKIADITIIIPYIGPALNIGNMLYKDDFVGALIFSGAVILLEFIPEIAIPVLGTFALVSYIANKVLTVQTIDNALSKRNEKWDEVYKYIVTNWLAKVNTQIDLIRKKMKEALENQAEATKAIINYQYNQYTEEEKNNINFNIDDLSSKLNESINKAMININKFLNQCSVSYLMNSMIPYGVKRLEDFDASLKDALLKYIYDNRGTLIGQVDRLKDKVNNTLSTDIPFQLSKYVDNQRLLSTFTEYIKNIINTSILNLRYESNHLIDLSRYASKINIGSKVNFDPIDKNQIQLFNLESSKIEVILKNAIVYNSMYENFSTSFWIRIPKYFNSISLNNEYTIINCMENNSGWKVSLNYGEIIWTLQDTQEIKQRVVFKYSQMINISDYINRWIFVTITNNRLNNSKIYINGRLIDQKPISNLGNIHASNNIMFKLDGCRDTHRYIWIKYFNLFDKELNEKEIKDLYDNQSNSGILKDFWGDYLQYDKPYYMLNLYDPNKYVDVNNVGIRGYMYLKGPRGSVMTTNIYLNSSLYRGTKFIIKKYASGNKDNIVRNNDRVYINVVVKNKEYRLATNASQAGVEKILSALEIPDVGNLSQVVVMKSKNDQGITNKCKMNLQDNNGNDIGFIGFHQFNNIAKLVASNWYNRQIERSSRTLGCSWEFIPVDDGWGERPL

SEQ ID NO: 2 - protein, 437 aa, synthetic construct
  source 1..437: note = Di-Chain Light-Chain 1
PFVNKQFNYKDPVNGVDIAYIKIPNAGQMQPVKAFKIHNKIWVIPERDTFTNPEEGDLNPPPEAKQVPVSYYDSTYLSTDNEKDNYLKGVTKLFERIYSTDLGRMLLTSIVRGIPFWGGSTIDTELKVIDTNCINVIQPDGSYRSEELNLVIIGPSADIIQFECKSFGHEVLNLTRNGYGSTQYIRFSPDFTFGFEESLEVDTNPLLGAGKFATDPAVTLAHELIHAGHRLYGIAINPNRVFKVNTNAYYEMSGLEVSFEELRTFGGHDAKFIDSLQENEFRLYYYNKFKDIASTLNKAKSIVGTTASLQYMKNVFKEKYLLSEDTSGKFSVDKLKFDKLYKMLTEIYTEDNFVKFFKVLNRKTYLNFDKAVFKINIVPKVNYTIYDGFNLRNTNLAANFNGQNTEINNMNFTKLKNFTGLFEFYKLLCVRGIITSK

SEQ ID NO: 3 - protein, 439 aa, synthetic construct
  source 1..439: note = Di-Chain Light-Chain 2
PFVNKQFNYKDPVNGVDIAYIKIPNAGQMQPVKAFKIHNKIWVIPERDTFTNPEEGDLNPPPEAKQVPVSYYDSTYLSTDNEKDNYLKGVTKLFERIYSTDLGRMLLTSIVRGIPFWGGSTIDTELKVIDTNCINVIQPDGSYRSEELNLVIIGPSADIIQFECKSFGHEVLNLTRNGYGSTQYIRFSPDFTFGFEESLEVDTNPLLGAGKFATDPAVTLAHELIHAGHRLYGIAINPNRVFKVNTNAYYEMSGLEVSFEELRTFGGHDAKFIDSLQENEFRLYYYNKFKDIASTLNKAKSIVGTTASLQYMKNVFKEKYLLSEDTSGKFSVDKLKFDKLYKMLTEIYTEDNFVKFFKVLNRKTYLNFDKAVFKINIVPKVNYTIYDGFNLRNTNLAANFNGQNTEINNMNFTKLKNFTGLFEFYKLLCVRGIITSKTK

SEQ ID NO: 4 - protein, 848 aa, synthetic construct
  source 1..848: note = Di-Chain Heavy-Chain
ALNDLCIKVNNWDLFFSPSEDNFTNDLNKGEEITSDTNIEAAEENISLDLIQQYYLTFNFDNEPENISIENLSSDIIGQLELMPNIERFPNGKKYELDKYTMFHYLRAQEFEHGKSRIALTNSVNEALLNPSRVYTFFSSDYVKKVNKATEAAMFLGWVEQLVYDFTDETSEVSTTDKIADITIIIPYIGPALNIGNMLYKDDFVGALIFSGAVILLEFIPEIAIPVLGTFALVSYIANKVLTVQTIDNALSKRNEKWDEVYKYIVTNWLAKVNTQIDLIRKKMKEALENQAEATKAIINYQYNQYTEEEKNNINFNIDDLSSKLNESINKAMININKFLNQCSVSYLMNSMIPYGVKRLEDFDASLKDALLKYIYDNRGTLIGQVDRLKDKVNNTLSTDIPFQLSKYVDNQRLLSTFTEYIKNIINTSILNLRYESNHLIDLSRYASKINIGSKVNFDPIDKNQIQLFNLESSKIEVILKNAIVYNSMYENFSTSFWIRIPKYFNSISLNNEYTIINCMENNSGWKVSLNYGEIIWTLQDTQEIKQRVVFKYSQMINISDYINRWIFVTITNNRLNNSKIYINGRLIDQKPISNLGNIHASNNIMFKLDGCRDTHRYIWIKYFNLFDKELNEKEIKDLYDNQSNSGILKDFWGDYLQYDKPYYMLNLYDPNKYVDVNNVGIRGYMYLKGPRGSVMTTNIYLNSSLYRGTKFIIKKYASGNKDNIVRNNDRVYINVVVKNKEYRLATNASQAGVEKILSALEIPDVGNLSQVVVMKSKNDQGITNKCKMNLQDNNGNDIGFIGFHQFNNIAKLVASNWYNRQIERSSRTLGCSWEFIPVDDGWGERPL

SEQ ID NO: 5 - protein, 10 aa, synthetic construct
  source 1..10: note = C-terminal L-chain Fragment 1
TKSLDKGYNK

SEQ ID NO: 6 - protein, 8 aa, synthetic construct
  source 1..8: note = C-terminal L-chain Fragment 2
SLDKGYNK